(19) United States
(12) Patent Application Publication
(10) Pub. No.: US 2019/0228843 A1
(43) Pub. Date: Jul. 25, 2019

(54) Molecule-Based Equation Oriented Reactor Simulation System And Its Model Reduction

(71) Applicant: Aspen Technology, Inc., Bedford, MA (US)

(72) Inventors: Zhen Hou, Lexington, MA (US); Darin Campbell, Waltham, MA (US)

(21) Appl. No.: 16/250,445

(22) Filed: Jan. 17, 2019

Related U.S. Application Data
(60) Provisional application No. 62/619,592, filed on Jan. 19, 2018.

(51) Int. Cl. G16C 20/10; G06F 17/50

(52) U.S. Cl. CPC G16C 20/10; G06F 17/5009

(57) ABSTRACT

A computer-implemented method and system for modeling chemical reaction in a chemical reactor is disclosed. The method and system employ a molecule-based equation-oriented approach. Local-stored pre-estimated thermodynamic properties are generated based on a set of homologous series of compounds defined by the method and system. A set of reaction rate equations is automatically generated in equation-oriented format based on the defined set of homologous series of compounds, a system-defined set of permissible reactions, system-defined properties of the reactor compounds, and the locally-stored pre-estimated thermodynamic properties. The automatically generated set of reaction rate equations forms the model of chemical reactions in the chemical reactor.

### RELATED APPLICATIONS

This application claims the benefit of U.S. Provisional Application No. 62/619,592, filed on Jan. 19, 2018. The entire teachings of the above application is incorporated herein by reference.

### BACKGROUND

Conventional, computer-implemented hydrocarbon process simulation modeling often describes the species in the refining models in terms of lumped pseudo components that are classified by physical properties (e.g. boiling point, solubility, carbon number, etc.). These classifications lack chemical structure. As a result, conventional models cannot describe the components beyond their lumps' definitions and lack information revealing the nature of chemical reactions in refining processes. Consequently, conventional models are unable to provide sufficiently high quality predictions of properties strongly associated with molecular structures (e.g., research octane number (RON), motor octane number (MON), and melting point). In addition, users cannot obtain deep understanding of refining chemistries and describe unprecedented feedstocks (e.g., biofuel and heavy residual) using the conventional lumped approach.

Modeling hydrocarbon conversions of complex chemistries at the molecular level often requires solving a large-scale problem including thousands of species and tens of thousands of reactions. The numerical computational burden is a challenging problem for developing a molecular level modeling software for complex mixtures and chemistries. Thermodynamic models for evaluating the properties involved in reactor simulations require an extremely large number of variables for the thousands of species within the model. Moreover, the computer memory required increases dramatically when the number of variables increases. Many conventional approaches require manual coding of reaction rate equations, which is very time consuming, and accordingly it is not practical to manually code all of the possible reaction rate equations that may occur in a given chemical reactor.

Accordingly, there is a need for improved computer-implemented methods of modeling chemical reactions in a chemical reactor, particularly for complex hydrocarbon processes.

### SUMMARY

Described herein is a computer-implemented method of modeling chemical reactions in a chemical reactor. Reactor compounds are represented by defining a set of homologous series of compounds in the reactor, each homologous series within the set comprising a molecular type and a carbon number range. A set of permissible reactions is defined for the defined set of homologous series of compounds. Properties of the reactor compounds are defined. Pre-estimated thermodynamic properties are generated and locally-stored. The pre-estimated thermodynamic properties are based on the defined set of homologous series of compounds. A set of reaction rate equations is automatically coded in equation oriented format based on: i) the defined set of homologous series of compounds; ii) the defined set of permissible reactions; iii) the defined properties of the reactor compounds; and iv) the generated and locally-stored pre-estimated thermodynamic properties. As a result, a model of chemical reactions in the chemical reactor is formed by the automatically coded set of reaction rate equations.

Described herein is a computer system for modeling chemical reactions in a chemical reactor. The computer system can include one or more processors operatively coupled to associated memory. The processors are configured to represent reactor compounds by defining a set of homologous series of compounds in the reactor, each homologous series within the set comprising a molecular type and a carbon number range. The processors define a set of permissible reactions for the defined set of homologous series of compounds. The processors are configured to define properties of the reactor compounds. The processors are configured to generate and locally-store pre-estimated thermodynamic properties based on the defined set of homologous series of compounds. The processors are programmed so as to automatically code a set of reaction rate equations in equation oriented format based on: i) the defined set of homologous series of compounds; ii) the defined set of permissible reactions; iii) the defined properties of the reactor compounds; and iv) the generated and locally-stored pre-estimated thermodynamic properties. As a result, a model of chemical reactions in the chemical reactor is formed by the automatically coded set of reaction rate equations.

Described herein is a computer program product that includes a computer readable medium carrying instructions that model chemical reactions in a chemical reactor. The instructions include computer code which when executed by a digital processor cause a simulator of the chemical reactor to implement the methods described herein.

In embodiments, the molecular type of the homologous series can be one or more of: molecular hydrogen (H₂); normal paraffin; pyrrole; benzene; pyridine; cyclohexane; thiophene; tetralin; benzothiophene; indole; naphthalene; quinolone; decalin; tetradecahydrophenanthrene; fluorene; tetrahydrophenanthrene; phenanthrene; benzoquinoline; octadecahydrochrysene; chrysene; naphthoquinoline; picene; naphthobenzothiophene (hard sulfur removal); naphthobenzothiophene (not as hard sulfur removal); dibenzothiophene (hard sulfur removal); dibenzothiophene (not as hard sulfur removal); carbazole; benzocarbazole; light molecule (<C4); tetrahydropyrrole; tetrahydrothiophene; dihydrobenzothiophene; dihydroindole; tetrahydroquinoline; octahydrophenanthrene; tetrahydrobenzoquinoline; tetrahydrochrysene; tetrahydronaphthoquinoline; tetrahydropicene; phenylnaphthalene; hydrogen sulfide; hexahydronaphthobenzothiophene; tetrahydronaphthobenzothiophene (hard sulfur removal); tetrahydronaphthobenzothiophene (not as hard sulfur removal); biphenyl; hexahydrodibenzothiophene; hexahydrocarbazole; hexahydrobenzocarbazole; tetrahydrobenzocarbazole; octahydrobenzothiophene; ammonia; octahydroindole; octahydrobenzoquinoline; octahydropicene; cyclohexylnaphthalene; decahydronaphthobenzothiophene; cyclohexylbenzene; dodecahydrodibenzothiophene; decahydronaphthobenzoquinoline; iso paraffin with one branch; tetradecahydrobenzoquinoline; hexahydrofluorene; iso paraffin with multiple branches; sulfide/mercaptan; tetrahydrobenzoquinoline; tetrahydronaphthoquinoline; octahydronaphthoquinoline; dodecahydrocarbazole; hexadecahydronaphthobenzothiophene; bicyclohexyl; hexadecahydronaphthoquinoline; cyclohexyldecalin; dodecahydronaphthoquinoline; naphthalene connected with chrysene; naphthalene connected with dibenzothiophene; phenanthrene connected with chrysene; phenanthrene connected with naphthobenzothiophene; phenanthrene connected with dibenzothiophene; chyrsene connected with chrysene; chyrsene connected with naphthobenzothiophene; chyrsene connected with picene; picene connected with picene; naphthobenzothiophene connected with picene; naphthobenzothiophene connected with naphthobenzothiophene; and dibenzothiophene connected with naphthobenzothiophene. In some embodiments, the molecular type of the homologous series is one or more of: normal paraffin; iso paraffin with one branch; and iso paraffin with multiple branches.

In some embodiments, the molecular type of the homologous series includes any combination of one or more of: naphthalene fused with naphthalene; naphthalene fused with benzothiophene; naphthalene fused with indole; naphthalene fused with quinoline; quinoline fused with quinoline; benzothiophene fused with quinoline; indole fused with quinoline; biphenyl fused with naphthalene; biphenyl fused with quinoline; benzothiophene fused with benzothiophene; benzothiophene fused with indole; biphenyl fused with benzothiophene; indole fused with indole; biphenyl fused with indole; phenanthrene fused with naphthalene; phenanthrene fused with quinoline; phenanthrene fused with benzothiophene; phenanthrene fused with indole; phenanthrene fused with phenanthrene; biphenyl fused with phenanthrene; benzoquinoline fused with quinoline; benzoquinoline fused with benzothiophene; benzoquinoline fused with indole; benzoquinoline fused with phenanthrene; benzoquinoline fused with benzoquinoline; biphenyl fused with benzoquinoline; dibenzothiophene (hard sulfur removal) fused with naphthalene; dibenzothiophene (hard sulfur removal) fused with quinoline; dibenzothiophene (hard sulfur removal) fused with benzothiophene; dibenzothiophene (hard sulfur removal) fused with indole; dibenzothiophene (hard sulfur removal) fused with phenanthrene; dibenzothiophene (hard sulfur removal) fused with benzoquinoline; dibenzothiophene (not hard sulfur removal) fused with naphthalene; dibenzothiophene (not hard sulfur removal) fused with benzoquinoline; dibenzothiophene (not hard sulfur removal) fused with benzothiophene; dibenzothiophene (not hard sulfur removal) fused with indole; dibenzothiophene (not hard sulfur removal) fused with phenanthrene; dibenzothiophene (not hard sulfur removal) fused with benzoquinoline; carbazole fused with naphthalene; carbazole fused with quinoline; carbazole fused with indole; carbazole fused with phenanthrene; carbazole fused with benzoquinoline; biphenyl fused with chrysene; biphenyl fused with naphthoquinoline; phenylnaphthalene fused with phenylnaphthalene; biphenyl fused with phenylnaphthalene; naphthobenzothiophene (hard sulfur removal) fused with phenylnaphthalene; chrysene fused with naphthalene; chrysene fused with quinoline; chrysene fused with benzothiophene; chrysene fused with indole; chrysene fused with phenanthrene; chrysene fused with benzoquinoline; chrysene fused with phenylnaphthalene; chrysene fused with benzothiophene1; chrysene fused with indole1; naphthoquinoline fused with quinoline; naphthoquinoline fused with benzothiophene; naphthoquinoline fused with indole; naphthoquinoline fused with benzoquinoline; naphthoquinoline fused with phenylnaphthalene; naphthoquinoline fused with benzothiophene1; naphthoquinoline fused with indole1; naphthobenzothiophene (hard sulfur removal) fused with benzothiophene; naphthobenzothiophene (hard sulfur removal) fused with indole; naphthobenzothiophene (hard sulfur removal) fused with naphthalene; naphthobenzothiophene (hard sulfur removal) fused with benzoquinoline; naphthobenzothiophene (hard sulfur removal) fused with biphenyl; naphthobenzothiophene (not hard sulfur removal) fused with benzothiophene; naphthobenzothiophene (not hard sulfur removal) fused with indole; naphthobenzothiophene (not hard sulfur removal) fused with naphthalene; naphthobenzothiophene (not hard sulfur removal) fused with benzoquinoline; naphthobenzothiophene (not hard sulfur removal) fused with phenylnaphthalene; benzocarbazole (not hard sulfur removal) fused with indole; benzocarbazole (not hard sulfur removal) fused with naphthalene; benzocarbazole (not hard sulfur removal) fused with quinoline; benzocarbazole (not hard sulfur removal) fused with phenylnaphthalene; picene fused with naphthalene; picene fused with quinoline; picene fused with biphenyl; naphthobenzothiophene (not hard sulfur removal) fused with biphenyl; picene fused with benzothiophene; picene fused with indole; picene fused with phenanthrene; picene fused with benzoquinoline; picene fused with phenylnaphthalene; picene fused with benzothiophene1; picene fused with indole1; picene fused with phenanthrene1; picene fused with benzoquinoline1; and picene fused with phenylnaphthalenel.

In some embodiments, the automatically coded set of reaction rate equations include Langmuir-Hinshelwood-Hougen-Watson (LHHW) rate laws. In some embodiments, generating and locally storing the pre-estimated thermodynamic properties includes solving equations of kinetic rate and constraining kinetic rate constants by a linear free energy relationship (LFER). In some embodiments, the set of reaction rate equations comprises one or more of residuals, sparsity patterns, and analytical Jacobians in equation-oriented format.

In some embodiments, the defined properties of the reactor compounds include one or more of: molecular weight; total number of carbon atoms; total number of hydrogen atoms; total number of side chains; total number of aromatic rings; total number of naphthenic rings; total number of thiophenic rings; total number of pyrrolic rings; total number of pyridenic rings; total number of sulfur atoms; total number of nitrogen atoms; total number of oxygen atoms; total number of aromatic carbon atoms; total number of naphthenic carbon atoms; total number of paraffinic carbon atoms; total number of naphthenic six-carbon rings; total number of naphthenic five-carbon rings; boiling point; density; standard enthalpy of formation in gas phase; standard Gibbs free energy of formation in gas phase; a gas phase heat capacity coefficient; heat of vaporization; standard enthalpy of formation in liquid phase; a liquid phase heat capacity coefficient; a viscosity coefficient; and molecular type. In one embodiment, the defined properties of reactor compounds are one or more of: total number of carbon atoms, total number of hydrogen atoms, total number of aromatic rings, total number of naphthenic rings, total number of thiophenic rings, total number of pyrrolic rings, total number of pyridenic rings, total number of sulfur atoms, total number of nitrogen atoms, total number of oxygen atoms, standard Gibbs free energy of formation, and standard enthalpy of formation. In another embodiment, the defined properties of reactor compounds are one or more of total number of carbon atoms, total number of hydrogen atoms, standard Gibbs free energy of formation, and standard enthalpy of formation.

In some embodiments, the generated and locally-stored pre-estimated thermodynamic properties include one or more of: enthalpy of formation in gas phase at given temperature; Gibbs free energy of formation in gas phase at given temperature; gas phase heat capacity at given temperature; entropy in gas phase at given temperature; heat of vaporization; enthalpy of formation in liquid phase at given temperature; and liquid phase heat capacity at given temperature. In one embodiment, the generated and locally-stored pre-estimated thermodynamic properties are one or more of: enthalpy of formation in gas phase at given temperature; Gibbs free energy of formation in gas phase at given temperature; gas phase heat capacity at given temperature; and entropy in gas phase at given temperature. In other embodiments, the generated pre-estimated thermodynamic properties are enthalpy of formation in liquid phase at given temperature; and liquid phase heat capacity at given temperature.

In some embodiments, the defined set of permissible reactions include one or more of: saturate a benzene ring in thiophenics with 3H₂; saturate a benzene ring in pyridinics or pyrrolics with 3H₂; saturate a benzene ring in pure hydrocarbon with 3H₂; saturate an isolated thiophenic ring with 2H₂; saturate a pyridinic ring fused with a benzene ring with 2H₂, or saturate an isolated pyrrolic ring with 2H₂; saturate a benzene ring in pure hydrocarbon with 2 H₂; saturate a thiophenic ring fused with a benzene ring with 1H₂; saturate a pyrrolic ring fused with a benzene ring with 1H₂; naphthenics ring opening; paraffin hydrocracking; paraffin isomerization; desulfurization of thiophenics; desulfurization of saturated thiophenics in saturated dibenzothiophene structures; desulfurization of saturated benzothiophene, or desulfurization of saturated thiophene structures; denitrogenation of saturated nitrogen rings in saturated carbazole structures; denitrogenation of saturated nitrogen rings in saturated indoles, pyrroles, pyridine, or quinine structures; dealkylation; and inter-core linkage cracking (ILCR). In one embodiment, the defined set of permissible reactions is paraffin isomerization. In one embodiment, the defined set of permissible reactions includes aromatic ring condensation.

In some embodiments, automatically coding a set of reaction rate equations comprises parsing a reaction into reactants, products, and stoichiometric coefficients for the reactants and products. In some embodiments, automatically coding a set of reaction rate equations includes generating one or more of a residual, a sparsity, and an analytical Jacobian.

In some embodiments, generating the locally stored pre-estimated thermodynamic properties is by solving one or more equations of mass balance, energy balance, momentum balance, and kinetic rate and their associated ordinary differential equations. In some embodiments, solving one or more equations of mass balance, energy balance, momentum balance, and kinetic rate and their associated ordinary differential equations further includes providing initial solutions of the one or more equations.

The method can also include outputting a table of results of the formed reactor model to a flowsheet simulator.

In some embodiments, the formed reactor model is considered a full reactor model. The method can further include creating a list of active species of the defined set of homologous series of compounds, and storing active species of the defined set of permissible reactions in the created list of active species, thereby creating a reduced reactor model from the full reactor model. In system embodiments, the processor can be further configured to create a list of active species of the defined set of homologous series of compounds, and store active species of the defined set of permissible reactions in the created list of active species, thereby creating a reduced reactor model from the full reactor model.

Any of the methods described herein can be implemented in computer systems, reactor simulation systems, refinery systems, and the like described herein.

The methods described herein provide a number of benefits compared to prior methods. The methods can be used to create a molecular level kinetic model for refining chemistries in equation-oriented format. A large number of components and reactions is supported (e.g., on the order of 10000 species and 50000 reactions). Notably, the formed reactor model of embodiments can be described in terms of the molecular components. Thus, the formed model provides an improved level of detail, or resolution, that is useful for predicting properties, such as yield, octane number (e.g., research octane number (RON) for gasoline), and cetane number for diesel fuel. In some embodiments, improved detail can also be used to quantify particular compounds, such as quantity or mole percent of benzene. In some embodiments, improved detail can also be used to quantify particular atoms of interest, such as the amount of sulfur, which is typically expressed in parts-per-million (PPM). Other properties of interest can include viscosity, pour point, freeze point, and aromatic content.

Prior methods required coding individual reaction rate equations, which is very time consuming and tedious. As a practical matter, coding individual reaction rate equations limits the number of equations that can be utilized due to the amount of time necessary to code the equations. Automatically coding a set of reaction rate equations by embodiments of the present invention greatly accelerates the development of a refining reactor model since it is often cost-prohibitive to manually code a large set of reaction rate equations.

In embodiments where the method further includes creating a reduced reactor model, an additional advantage includes allowing users to simulate a large-scale molecule-based kinetic problem in terms of a molecule-based reduced model. In particular, the number of numerical variables is reduced to a smaller size, which further reduces memory and computational requirements (processing resources). Since there are fewer variables, the solution time of the reduced model for multiple beds is shorter while maintaining full molecular details for the reactor beds.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1 is a schematic diagram of a computer-implemented method of modeling chemical reactions in a chemical reactor in embodiments.

FIG. 2A is a diagram depicting how a model builder in embodiments reads input data and generates equations in equation-oriented (EO) format for a molecular-based (MB) Reactor model. FIG. 2B is a description of coded equations in equation-oriented format.

FIG. 3 is a table depicting an example of a set of homologous series of compounds in embodiments.

FIG. 4A is a table that depicts examples of permissible reactions that can occur within a reactor as defined by embodiments. FIG. 4B is a table that depicts examples of permissible reactions that can occur within a reactor as defined by embodiments.

FIG. 5 is a flow diagram of a model reduction embodiment controlling size of a generated molecular-based reactor model.

FIG. 6 is a table depicting an example set of homologous series of compounds and model reduction zoning in a homologous series of hydrocarbon molecular compositions in an embodiment.

FIG. 7 is a chart showing comparison of the distribution of mole fractions versus boiling point between a molecule based full hydrocracker model embodiment and a molecule based reduced hydrocracker model embodiment.

FIG. 8 is a parity plot of the mole fractions profile between the molecule based full hydrocracker model embodiment and the molecule based reduced hydrocracker model embodiment, both of FIG. 7.

FIG. 9 is a schematic illustration of a computer network in which embodiments operate.

FIG. 10 is a block diagram of one computer node in the computer network of FIG. 9.

### DETAILED DESCRIPTION

A description of example embodiments follows.

### Overview

The traditional approach to modeling refinery processes involves lumping, or grouping, molecules together that have similar properties, such as boiling point. For modeling reactor processes, molecules are lumped together by molecule-type species. It is difficult or impossible to come up with a set of lumps that can adequately model both fractionation and reactor processes, such as fluid catalytic cracking and hydrocracking. Greater resolution is desirable because it can permit more refined prediction of the properties of the resulting material and the ability to model both fractionator and reactor processes with a common set of components.

The methods described herein employ an equation-oriented (EO) approach. Instead of solving each equation sequentially, an equation-oriented approach solves the set of equations simultaneously. Thus, equation-oriented modeling is sometimes called equation-based or simultaneous equation modeling. A brief introduction of EO is shown as Eq. 1 to Eq. 2. Traditionally, a reaction rate is expressed by sequential module (SM) method as:

$$\frac{{dC}_{A}}{d τ} = {r}_{A} ( k , T , P , {C}_{A} ) \qquad Eq . 1$$

The EO format of the reaction rate of Equation 1 is shown as the following:

$${f}_{res} = \frac{{dC}_{A}}{d τ} - {r}_{A} ( k , T , P , {C}_{A} ) = 0 \qquad Eq . 2$$

The model in EO essentially is to solve Eq. 2. One advantage of the EO approach is the flexibility to switch the independent variables. We can use either

$$\frac{{dC}_{A}}{d τ} ,$$

k,T,P, C_{A} as the input variables without rewriting the equation to change the model structure. To obtain the optimal solving performance, the sparsity and the jacobians of all variables

$$( \frac{{dC}_{A}}{d τ} ,$$

k,T,P, C_{A}) to the residual function (ƒᵣₑₛ) in Eq. 2 need to be specified.

The methods described herein refer to coding a set of reaction rate equations, which are of the form or format suitable for an equation-oriented approach.

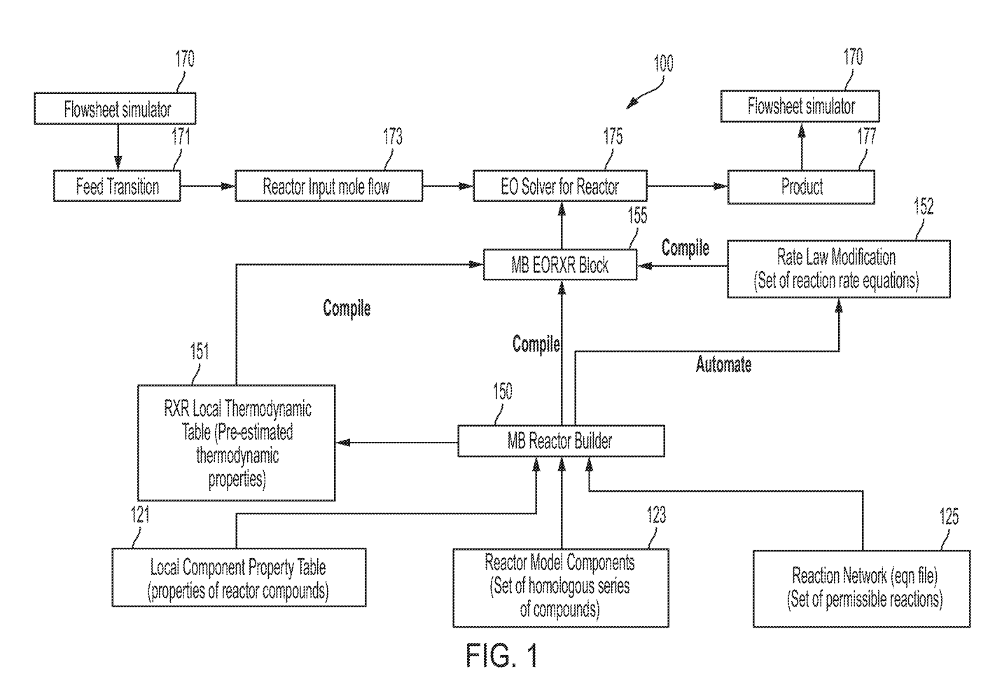

FIG. 1 is a schematic diagram of a computer-implemented method and system 100 of embodiments modeling chemical reactions in a chemical reactor.

“Reactor model components” module 123 defines representative core compounds that are expected to be present in the subject reactor, either as reactants, products, or reaction intermediates. The module 123 represents and describes reactor model components (compounds) with respect to the homologous series of compounds. In many cases, thousands of reactor model compounds are defined by module 123.

In response to the set of reactor model components defined at module 123, a reaction network 125 identifies and defines a set of permissible reactions for the defined set of homologous series of compounds. In turn, the reaction network 125 forms a file of equations representing the permissible reactions in the EO format illustrated by Eq. 2.

A “Local component property” module 121 provides tables or data defining properties of the reactor compounds. Such property data tables or information are further described below with respect to Table 3.

The reactor model components from module 123, reaction network equations (set of permissible reactions) from module 125, and local component property information (properties of reactor compounds) from module 121 are input into a molecular-based (MB) Reactor Builder 150 to generate the equations of an MB reactor model. In particular, the MB Reactor Builder 150 pre-estimates thermodynamic properties of the reactor compounds by solving equations of mass balance, energy balance, momentum balance, and kinetic rates and their associated ordinary differential equations. Next, the MB Reactor Builder 150 locally stores the generated pre-estimated thermodynamic properties at 151.

The MB Reactor Builder 150 also automatically codes a set of reaction rate equations at 152 based on the input from modules 121, 123 and network 125. MB Reactor Builder 150 compiles the locally-stored pre-estimated thermodynamic properties 151, and coded set of reaction rate equations 152 into the MB EORXR Block 155. Effectively, the MB EORXR Block 155 holds an EO formatted molecular-based (MB) model of the chemical reactions of the subject reactor formed of the coded set of reaction rate equations 152.

An EO Solver 175 solves the equations that define the MB reactor model from block 155. For a non-limiting example, the EO solver 175 may be part of a flowsheet simulator 170, such as HYSYS®, available from Aspen Technology (Bedford, Mass., USA). The flowsheet simulator 170 detects and tracks feed transitions 171 of the subject reactor. The flowsheet simulator 170 represents reactor input 173 in units of mole flows.

The EO solver 175 is responsive to the represented reactor input flow 173 and solves the MB reactor model equations from block 155. Restated, EO Solver 175 utilizes the reactor input flow values 173 of a flowsheet to set parameters and/or variables of the MB reactor model equations received as input from block 155.

The results (output) of the EO solver 175 are indications of predicted product 177 output by the modeled subject reactor. The product indications 177 include physical properties, other qualities, and quantities of the predicted materials output from the simulated reactor process. The flowsheet simulator 170 presents the product indications 177 as output or results of the simulation run.

### Homologous Series of Compounds

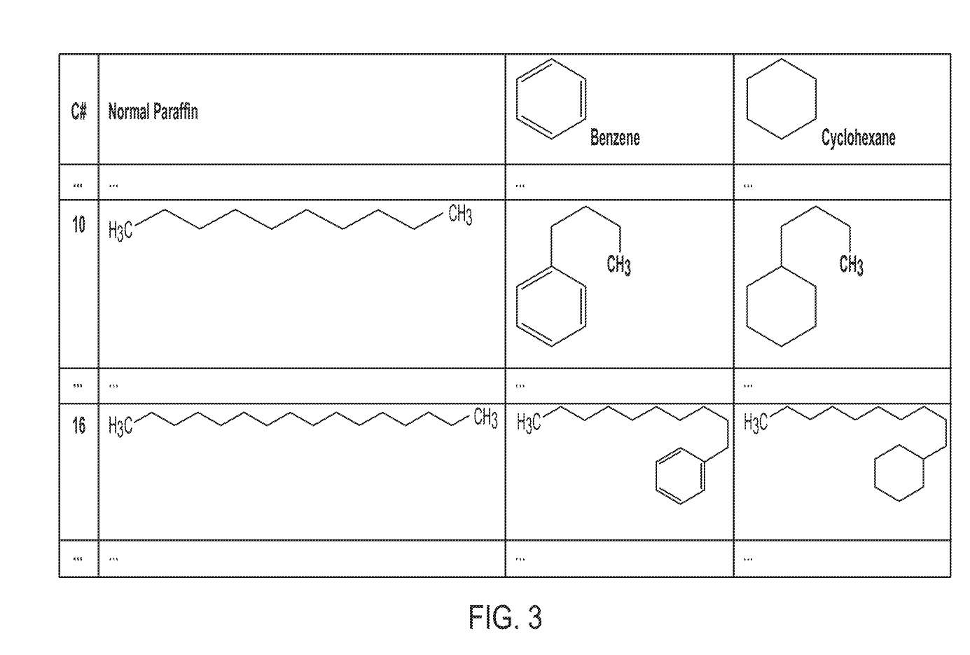

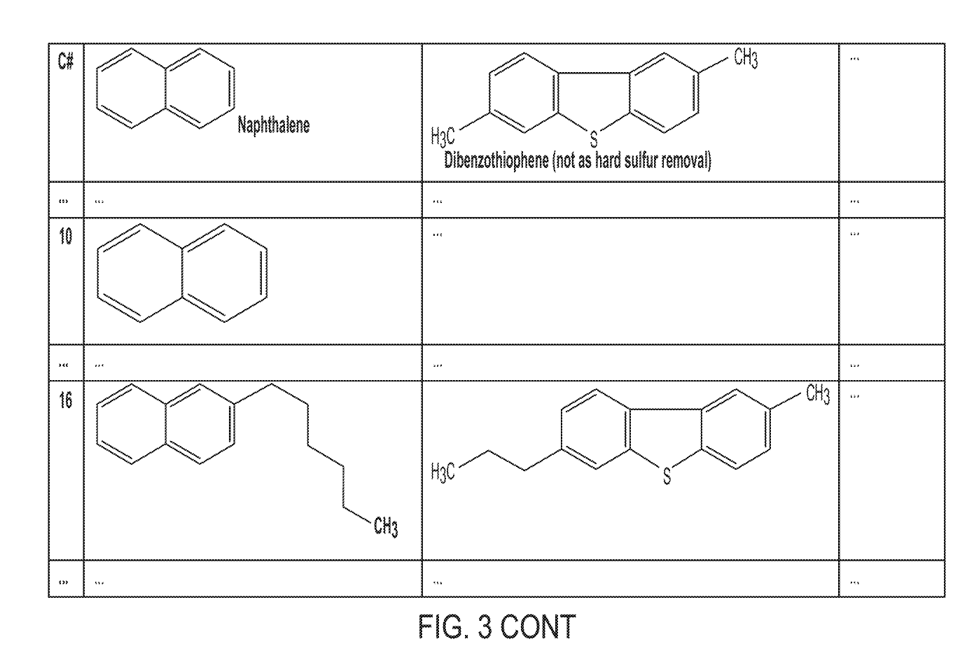

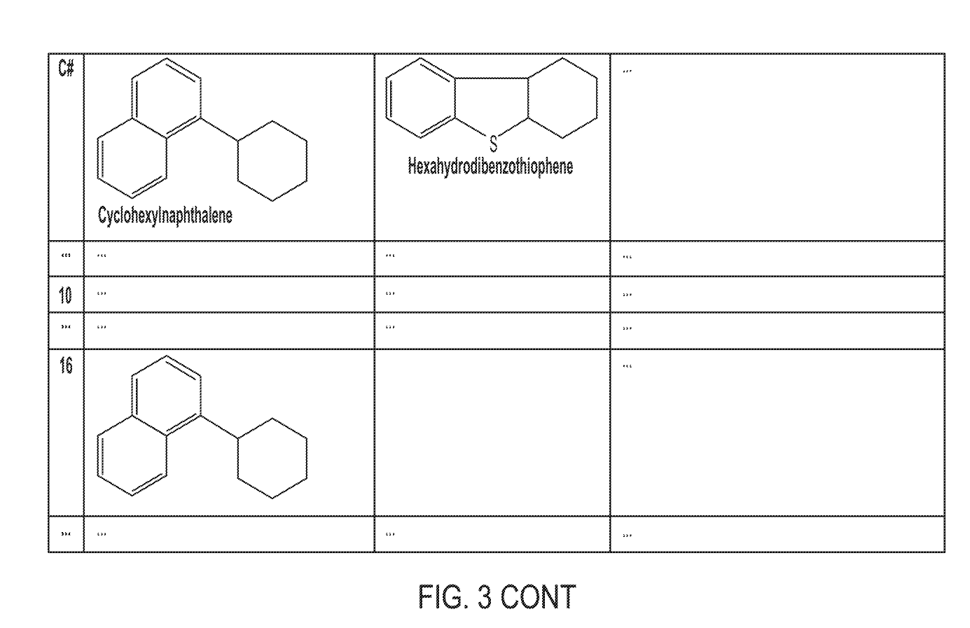

FIG. 3 is a table depicting an example of a set of homologous series of compounds defined by module 123 in embodiments. The individual compounds of the homologous series can be reactants, products, or reaction intermediates. In some embodiments, the set of homologous series of compounds can include about 2,000 species.

Each column represents one series that includes a unique molecular type, identified in the top row and sometimes referred to as the core compounds. The molecular types are sometimes referred to as “core” structures because the “core” is the unique aspect of the molecule in the column. In general, the molecular types contribute to reactivity and thermodynamic properties, and thus influence product quality and yields of hydrocarbon conversions. The molecular types identified in FIG. 3 are paraffin, benzene, cyclohexane, naphthalene, dibenzothiophene (not as hard sulfur removal), cyclohexylnaphthalene, and hexahydrodibenzothiophene. In the examples provided, there are between 76 to 90 molecular types (core structures).

Each row of FIG. 3 is a continuous carbon number extension for the corresponding molecular type. For example, in the paraffin molecule type, the row for 10 carbons is a 10-carbon linear paraffin, and the row for 16 carbons is a 16-carbon linear paraffin. For the benzene molecule type, the row for 10 carbons is a benzene ring (six carbons) with a four-carbon linear alkyl group, and the row for 16 carbons in a benzene ring (six carbons) with a ten-carbon linear alkyl group. For larger molecule types, there may not be an entry for a row that has fewer than the minimum required number of carbon atoms. For example, in the cyclohexylnaphthalene molecule type, the row for 10 carbons does not have an entry because the minimum number of required carbons is 16.

The juxtapositions of the molecular types and carbon numbers give a representation of the molecular composition of a hydrocarbon mixture. The left portion of FIG. 3 represents chemical compounds that are more commonly found in a feedstock in the hydrocracker. If desired, analytical assessments of the feedstock can be performed, and the molecular type and carbon number can be adjusted for the particular feedstock. Starting from the feedstock's molecular composition, the molecular composition of the hydrocracker reaction products are determined from hydrocracker reactions and kinetics, as described more fully with respect to FIG. 3. In FIG. 3, columns for paraffin, benzene, cyclohexane, naphthalene, dibenzothiophene (not as hard sulfur removal) are frequently molecular species in feedstock. The columns labeled cyclohexylnaphthalene, and hexahydrodibenzothiophene are Extended Molecular Species from Products, which represent particular chemical compounds that may result from a defined set of chemical reactions.

Tables 1.1 and 1.2 are non-exclusive lists of molecular types that may serve as the core structure for a homologous series of compounds in embodiments defined by module 123. In most embodiments, the core structures can have carbon atoms bonded to the core at a wide variety of positions. For example, the pyrrole molecular type can have additional carbon atoms bonded at any ring carbon. The cyclohexane molecular type can have a linear or branched alkyl chain attached to a single carbon of the cyclohexane. The cyclohexane molecular type can also have a linear or branched alkyl chain attached bonded to multiple different carbons of the cyclohexane core structure.

Some of the thiophene compounds of Tables 1.1 and 1.2 are referred to as “hard sulfur removal” and others are referred to as “not as hard sulfur removal.” Without wishing to be bound by theory, the molecular types referred to as “hard sulfur removal” include an aromatic ring bonded to opposite sides of the thiophene ring, and at least one R-group at either of the indicated positions. These R-groups sterically hinder interactions between a catalyst and the molecule, thereby impeding catalytic desulfurization. A wide variety of R-groups can provide steric hindrance, such as alkyl substituents. The R-groups themselves are typically stable in the sense that the rate of dealkylation at the indicated position is insubstantial. Carbon atoms can be bonded to the core structure elsewhere, so long as there is at least one R-group at either of the indicated positions. The molecular types referred to as “not as hard sulfur removal” can have R-groups at positions other than those indicated for the molecular types referred to as “hard sulfur removal.” The structures depicted in Tables 1.1 and 1.2 are examples of the locations where those R-groups may be located.

TABLE 1.1
Molecular types and associated structures.

| Species | | Description |
|---|---|---|
| ID | Structure | (Molecular Type) |
| H2 | H₂ | Molecular hydrogen |
| | | (H₂) |
| NP | 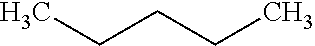 | normal paraffin |
| NN1 | 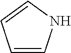 | pyrrole |
| A6_1 | 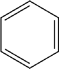 | Benzene |
| A6_1BN1 | 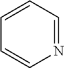 | Pyridine |
| N1 | 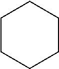 | Cyclohexane |
| ET1 | 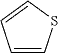 | Thiophene |
| A6_1N1 | 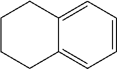 | Tetralin |
| A6_1ET1 | 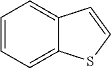 | Benzothiophene |
| A6_1NN1 | 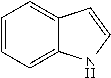 | Indole |
| A6_1A4_1 | 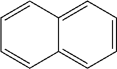 | Naphthalene |
| A6_1A4_1BN1 | 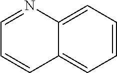 | Quinolone |
| N2 | 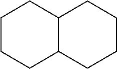 | Decalin |
| N3 | 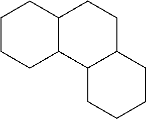 [SMILES: C1CCC2C(C1)CCC1CCCCC12] | Tetradecahydrophenan- threne |
| A6_2N1Ph1 | 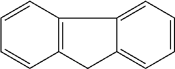 [SMILES: c1ccc2c(c1)Cc1ccccc1-2] | Fluorene |
| A6_1A4_1N1 | 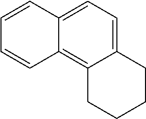 [SMILES: c1ccc2c3c(ccc2c1)CCCC3] | Tetrahydrophenanthrene |
| A6_1A4_2 | 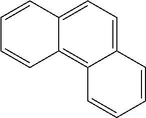 [SMILES: c1ccc2c(c1)ccc1ccccc12] | Phenanthrene |
| A6_1A4_2BN1 | 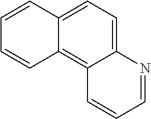 [SMILES: c1ccc2c(c1)ccc1ncccc12] | Benzoquinoline |
| N4 | 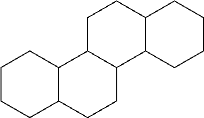 [SMILES: C1CCC2C(C1)CCC1C3CCCCC3CCC21] | Octadecahydrochrysene |
| A6_1A4_3 | 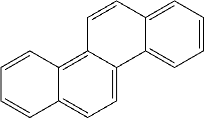 [SMILES: c1ccc2c(c1)ccc1c3ccccc3ccc21] | Chrysene |
| A6_1A4_3BN1 | 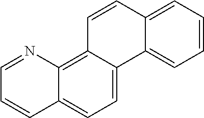 [SMILES: c1ccc2c(c1)ccc1c2ccc2cccnc21] | Naphthoquinoline |
| A6_1A4_4 | 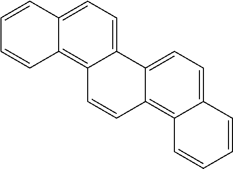 [SMILES: c1ccc2c(c1)ccc1c2ccc2c3ccccc3ccc21] | Picene |
| A6_2A4_1Ph1HT1 | 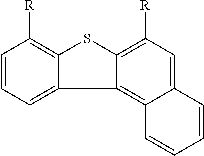 | Naphthobenzothiophene (hard sulfur removal) |
| A6_2A4_1Ph1ET1 | 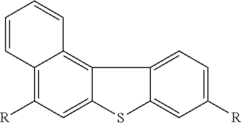 | Naphthobenzothiophene (not as hard sulfur removal) |
| A6_2Ph1HT1 | 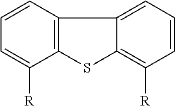 | Dibenzothiophene (hard sulfur removal) |
| A6_2Ph1ET1 | 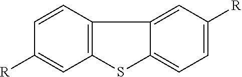 | Dibenzothiophene (not as hard sulfur removal) |
| A6_2Ph1NN1 | 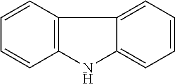 [SMILES: c1ccc2c(c1)[nH]c1ccccc12] | Carbazole |
| A6_2A4_1Ph1NN1 | 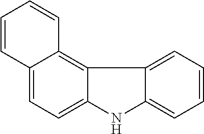 [SMILES: c1ccc2c(c1)ccc1[nH]c3ccccc3c12] | Benzocarbazole |
| LE | CH₄ | Light molecule (<C4) |
| SN1 | 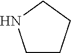 | Tetrahydropyrrole |
| SS1 | 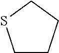 | Tetrahydrothiophene |
| A6_1SS1 | 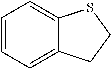 | Dihydrobenzothiophene |
| A6_1SN1 | 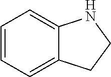 | Dihydroindole |
| A6_1N1BN1 | 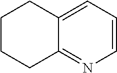 | Tetrahydroquinoline |
| A6_1N2 | 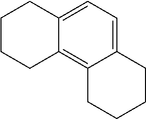 [SMILES: c1cc2c(c3c1CCCC3)CCCC2] | Octahydrophenanthrene |
| A6_1A4_1N1BN1 | 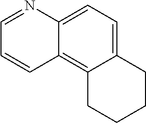 [SMILES: c1cnc2ccc3c(c2c1)CCCC3] | Tetrahydrobenzoquino- line |
| A6_1A4_2N1 | 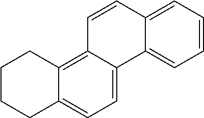 [SMILES: c1ccc2c(c1)ccc1c3c(ccc12)CCCC3] | Tetrahydrochrysene |
| A6_1A4_2N1BN1 | 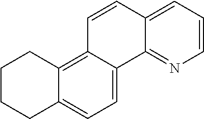 [SMILES: c1cnc2c(c1)ccc1c3c(ccc12)CCCC3] | Tetrahydronaphtho- quinoline |
| A6_1A4_3N1 | 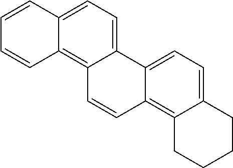 [SMILES: c1ccc2c(c1)ccc1c3ccc4c(c3ccc21)CCCC4] | Tetrahydropicene |
| A6_2A4_1Ph1 | 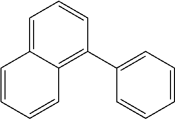 [SMILES: c1ccc(-c2cccc3ccccc23)cc1] | Phenylnaphthalene |
| H2S | SH₂ | Hydrogen sulfide |
| A6_1A4_1N1Ph1SS1 | 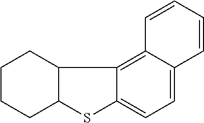 [SMILES: c1ccc2c3c(ccc2c1)SC1CCCCC31] | Hexahydronaphthobenzo- thiophene |
| A6_2N1Ph1HT1 | 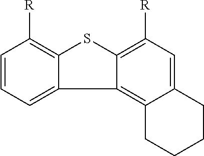 | Tetrahydronaphthobenzo- thiophene (hard sulfur removal) |
| A6_2N1Ph1ET1 | 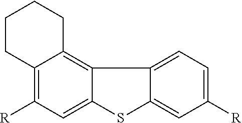 | Tetrahydronaphthobenzo- thiophene (not as hard sulfur removal) |
| A6_2Ph1 | 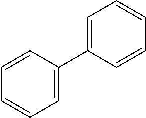 [SMILES: c1ccc(-c2ccccc2)cc1] | Biphenyl |
| A6_1N1Ph1SS1 | 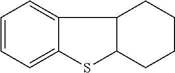 [SMILES: c1ccc2c(c1)SC1CCCCC21] | Hexahydrodibenzothio- phene |
| A6_1N1Ph1SN1 | 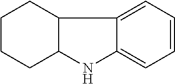 [SMILES: c1ccc2c(c1)NC1CCCCC21] | Hexahydrocarbazole |
| A6_1A4_1N1Ph1SN1 | 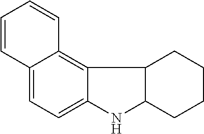 [SMILES: c1ccc2c3c(ccc2c1)NC1CCCCC31] | Hexahydrobenzocarba- zole |
| A6_2N1Ph1NN1 | 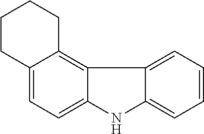 [SMILES: c1ccc2c(c1)[nH]c1ccc3c(c12)CCCC3] | Tetrahydrobenzocarba- zole |
| N1SS1 | 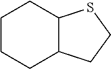 | Octahydrobenzothiophene |
| NH3 | NH₃ | Ammonia |
| N1SN1 | 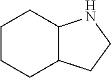 | Octahydroindole |
| A6_1N1SN1 | 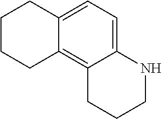 [SMILES: c1cc2c(c3c1CCCC3)CCCN2] | Octahydrobenzoquinoline |
| A6_1A4_2N2 | 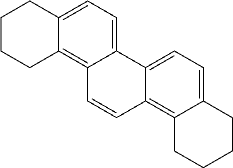 [SMILES: c1cc2c(ccc3c4c(ccc32)CCCC4)c2c1CCCC2] | Octahydropicene |
| A6_1A4_1N1Ph1 | 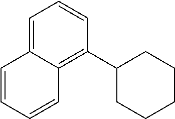 [SMILES: c1ccc2c(C3CCCCC3)cccc2c1] | Cyclohexylnaphthalene |
| A6_1N2Ph1SS1 | 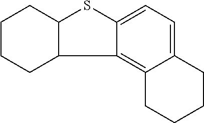 [SMILES: c1cc2c(c3c1CCCC3)C1CCCCC1S2] | Decahydronaphthobenzo- thiophene |
| A6_1N1Ph1 | 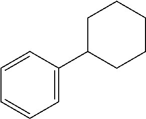 [SMILES: c1ccc(C2CCCCC2)cc1] | Cyclohexylbenzene |
| N2Ph1SS1 | 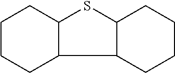 [SMILES: C1CCC2C(C1)SC1CCCCC12] | Dodecahydrodibenzo- thiophene |
| A6_1N2Ph1SN1 | 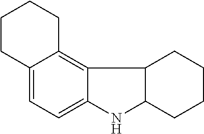 [SMILES: c1cc2c(c3c1CCCC3)C1CCCCC1N2] | Decahydronaphthobenzo- quinoline |
| SBP | 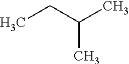 | Iso paraffin with one branch |
| N2SN1 | 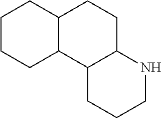 [SMILES: C1CCC2C(C1)CCC1NCCCC12] | Tetradecahydrobenzo- quinoline |
| A6_1N2Ph1 | 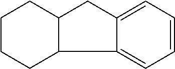 [SMILES: c1ccc2c(c1)CC1CCCCC21] | Hexahydrofluorene |
| MBP | 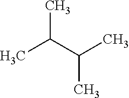 | Iso paraffin with multiple branches |
| HS | 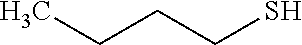 | Sulfide/mercaptan |
| A6_1A4_1SN1 | 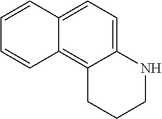 [SMILES: c1ccc2c3c(ccc2c1)NCCC3] | Tetrahydrobenzoquino- line |
| A6_1A4_2SN1 | 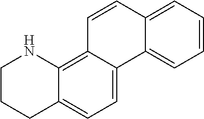 [SMILES: c1ccc2c(c1)ccc1c3c(ccc12)CCCN3] | Tetrahydronaphthoquin- oline |
| A6_1A4_1N1SN1 | 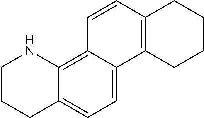 [SMILES: c1cc2c3c(ccc2c2c1CCCC2)CCCN3] | Octahydronaphthoquin- oline |
| N2Ph1SN1 | 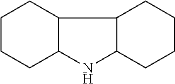 [SMILES: C1CCC2C(C1)NC1CCCCC12] | Dodecahydrocarbazole |
| N3Ph1SS1 | 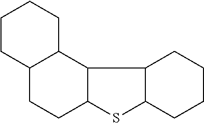 [SMILES: C1CCC2C(C1)CCC1SC3CCCCC3C12] | Hexadecahydronaphtho- benzothiophene |
| N2Ph1 | 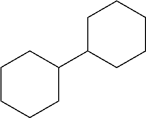 [SMILES: C1CCC(C2CCCCC2)CC1] | Bicyclohexyl |
| N3Ph1SN1 | 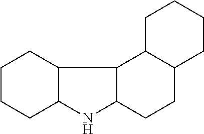 [SMILES: C1CCC2C(C1)CCC1NC3CCCCC3C12] | Hexadecahydronaphtho- quinoline |
| N3Ph1 | 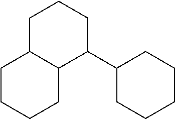 [SMILES: C1CCC(C2CCCC3CCCCC32)CC1] | Cyclohexyldecalin |
| A6_1N2SN1 | 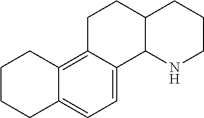 [SMILES: c1cc2c(c3c1CCCC3)CCC1CCCNC21] | Dodecahydronaphtho- quinoline |
| A6_2A4_4 | 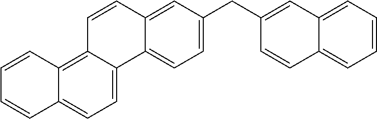 [SMILES: c1ccc2cc(Cc3ccc4c(ccc5c6ccccc6ccc45)c3)ccc2c1] | Naphthalene connected with chrysene |
| A6_3A4_1Ph1HT1 | 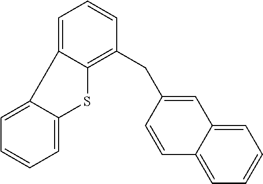 [SMILES: c1ccc2cc(Cc3cccc4c3sc3ccccc34)ccc2c1] | Naphthalene connected with dibenzothiophene |
| A6_2A4_5 | 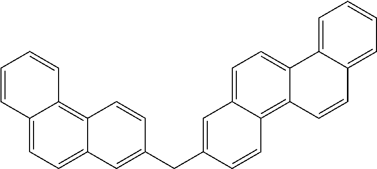 [SMILES: c1ccc2c(c1)ccc1cc(Cc3ccc4c(ccc5c6ccccc6ccc45)c3)ccc12] | Phenanthrene connected with chrysene |
| A6_3A4_3Ph1HT1 | 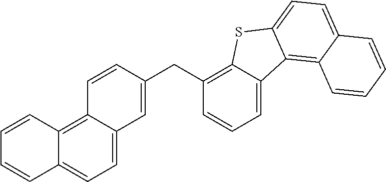 [SMILES: c1ccc2c(c1)ccc1cc(Cc3cccc4c3sc3ccc5ccccc5c34)ccc12] | Phenanthrene connected with naphthobenzothiophene |
| A6_3A4_2Ph1HT1 | 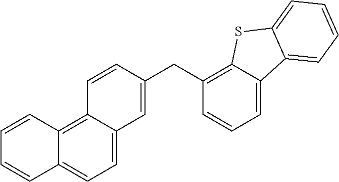 [SMILES: c1ccc2c(c1)ccc1cc(Cc3cccc4c3sc3ccccc34)ccc12] | Phenanthrene connected with dibenzothiophene |
| A6_2A4_6 | 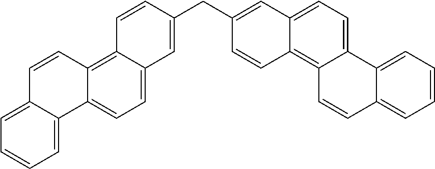 [SMILES: c1ccc2c(c1)ccc1c3ccc(Cc4ccc5c(ccc6c7ccccc7ccc56)c4)cc3ccc21] | Chyrsene connected with chrysene |
| A6_3A4_4Ph1HT1 | 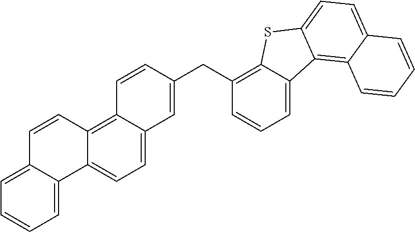 [SMILES: c1ccc2c(c1)ccc1c3ccc(Cc4cccc5c4sc4ccc6ccccc6c45)cc3ccc21] | Chyrsene connected with naphthobenzothiophene |
| A6_2A4_7 | 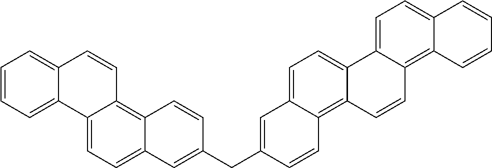 [SMILES: c1ccc2c(c1)ccc1c3ccc(Cc4ccc5c(ccc6c5ccc5c7ccccc7ccc56)c4)cc3ccc21] | Chyrsene connected with picene |
| A6_2A4_8 | 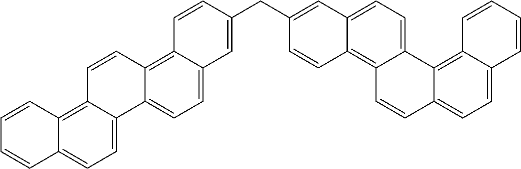 [SMILES: c1ccc2c(c1)ccc1c2ccc2c3ccc(Cc4ccc5c(ccc6c5ccc5ccc7ccccc7c56)c4)cc3ccc21] | Picene connected with picene |
| A6_3A4_5Ph1HT1 | 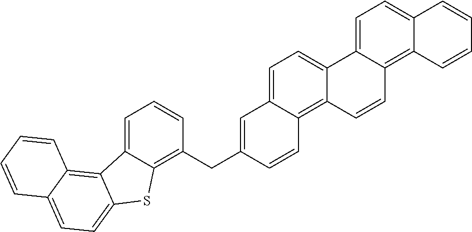 [SMILES: c1ccc2c(c1)ccc1c2ccc2c3ccc(Cc4cccc5c4sc4ccc6ccccc6c45)cc3ccc21] | Naphthobenzothiophene connected with picene |
| A6_4A4_2Ph2HT2 | 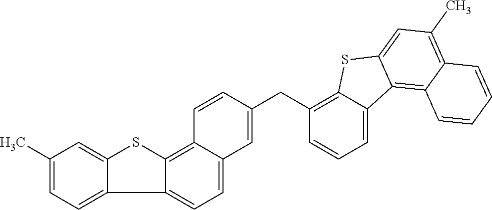 [SMILES: Cc1ccc2c(c1)sc1c3ccc(Cc4cccc5c4sc4cc(C)c6ccccc6c45)cc3ccc21] | Naphthobenzothiophene connected with naphthobenzothiophene |
| A6_4A4_1Ph2HT2 | 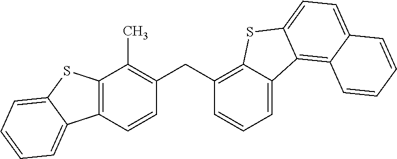 [SMILES: Cc1c(Cc2cccc3c2sc2ccc4ccccc4c23)ccc2c1sc1ccccc12] | Dibenzothiophene connected with naphthobenzothiophene |

TABLE 1.2
Molecular types and associated structures.

| Species | | Description |
|---|---|---|
| ID | Structure | (Molecular Type) |
| A6_2A4_2N1Ph2 | 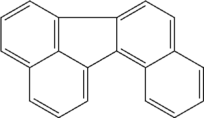 [SMILES: c1ccc2c3c(ccc2c1)-c1cccc2cccc-3c12] | Naphthalene fused with naphthalene |
| A6_2A4_1N1Ph2ET1 | 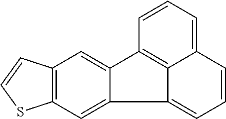 [SMILES: c1cc2c3c(cccc3c1)-c1cc3sccc3cc1-2] | Naphthalene fused with benzothiophene |
| A6_2A4_1N1Ph2NN1 | 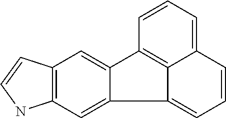 [SMILES: c1cc2c3c(cccc3c1)-c1cc3[nH]ccc3cc1-2] | Naphthalene fused with indole |
| A6_2A4_2N1Ph2BN1 | 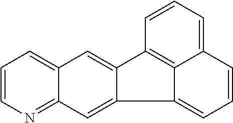 [SMILES: c1cnc2cc3c(cc2c1)-c1cccc2cccc-3c12] | Naphthalene fused with quinoline |
| A6_2A4_2N1Ph2BN2 | 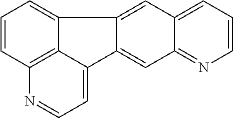 [SMILES: c1cnc2cc3c(cc2c1)-c1cccc2nccc-3c12] | Quinoline fused with quinoline |
| A6_2A4_1N1Ph2ET1BN1 | 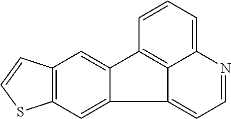 [SMILES: c1cc2c3c(ccnc3c1)-c1cc3sccc3cc1-2] | Benzothiophene fused with quinoline |
| A6_2A4_1N1Ph2BN1NN1 | 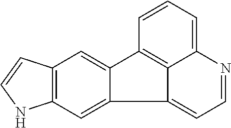 [SMILES: c1cc2c3c(ccnc3c1)-c1cc3[nH]ccc3cc1-2] | Indole fused with quinoline |
| A6_3A4_1N1Ph3 | 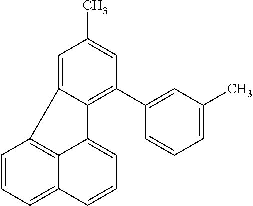 [SMILES: Cc1cccc(-c2cc(C)cc3c2-c2cccc4cccc-3c24)c1] | Biphenyl fused with naphthalene |
| A6_3A4_1N1Ph3BN1 | 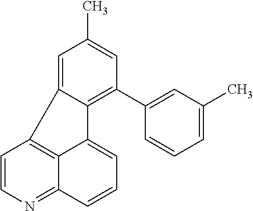 [SMILES: Cc1cccc(-c2cc(C)cc3c2-c2cccc4nccc-3c24)c1] | Biphenyl fused with quinoline |
| A6_2N1Ph2ET2 | 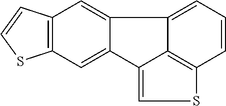 [SMILES: c1cc2c3c(csc3c1)-c1cc3sccc3cc1-2] | Benzothiophene fused with benzothiophene |
| A6_2N1Ph2ET1NN1 | 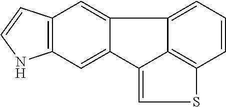 [SMILES: c1cc2c3c(csc3c1)-c1cc3[nH]ccc3cc1-2] | Benzothiophene fused with indole |
| A6_3N1Ph3ET1 | 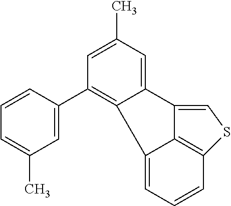 [SMILES: Cc1cccc(-c2cc(C)cc3c2-c2cccc4scc-3c24)c1] | Biphenyl fused with benzothiophene |
| A6_2N1Ph2NN2 | 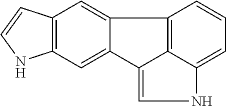 [SMILES: c1cc2c3c(c[nH]c3c1)-c1cc3[nH]ccc3cc1-2] | Indole fused with indole |
| A6_3N1Ph3NN1 | 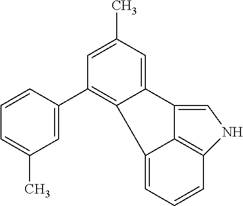 [SMILES: Cc1cccc(-c2cc(C)cc3c2-c2cccc4[nH]cc-3c24)c1] | Biphenyl fused with Indole |
| A6_2A4_3N1Ph2 | 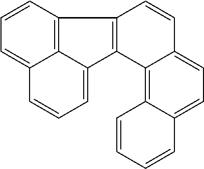 [SMILES: c1cc2c3c(cccc3c1)-c1c-2ccc2ccc3ccccc3c12] | Phenanthrene fused with naphthalene |
| A6_2A4_3N1Ph2BN1 | 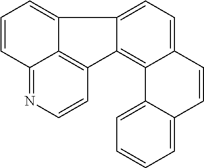 [SMILES: c1ccc2c(c1)ccc1ccc3c(c12)-c1ccnc2cccc-3c12] | Phenanthrene fused with quinoline |
| A6_2A4_2N1Ph2ET1 | 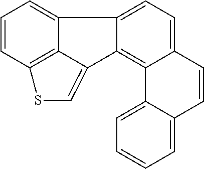 [SMILES: c1ccc2c(c1)ccc1ccc3c(c12)-c1csc2cccc-3c12] | Phenanthrene fused with benzothiophene |
| A6_2A4_2N1Ph2NN1 | 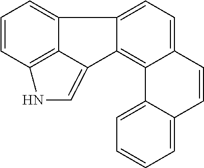 [SMILES: c1ccc2c(c1)ccc1ccc3c(c12)-c1c[nH]c2cccc-3c12] | Phenanthrene fused with indole |
| A6_2A4_4N1Ph2 | 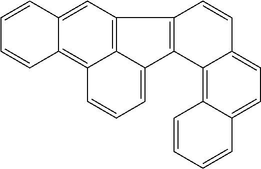 [SMILES: c1ccc2c(c1)cc1c3c(cccc32)-c2c-1ccc1ccc3ccccc3c21] | Phenanthrene fused with Phenanthrene |
| A6_3A4_2N1Ph3 | 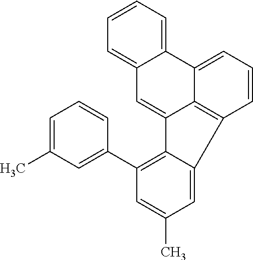 [SMILES: Cc1cccc(-c2cc(C)cc3c2-c2cc4ccccc4c4cccc-3c24)c1] | Biphenyl fused with Phenanthrene |
| A6_2A4_3N1Ph2BN2 | 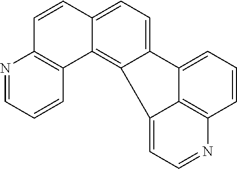 [SMILES: c1cc2c3c(ccnc3c1)-c1c-2ccc2ccc3ncccc3c12] | Benzoquinoline fused with quinoline |
| A6_2A4_2N1Ph2ET1BN1 | 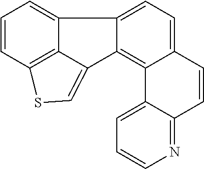 [SMILES: c1cc2c3c(csc3c1)-c1c-2ccc2ccc3ncccc3c12] | Benzoquinoline fused with benzothiophene |
| A6_2A4_2N1Ph2BN1NN1 | 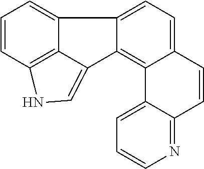 [SMILES: c1cc2c3c(c[nH]c3c1)-c1c-2ccc2ccc3ncccc3c12] | Benzoquinoline fused with indole |
| A6_2A4_4N1Ph2BN1 | 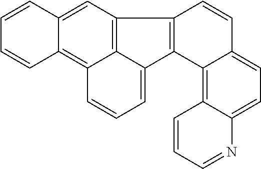 [SMILES: c1ccc2c(c1)cc1c3c(cccc32)-c2c-1ccc1ccc3ncccc3c21] | Benzoquinoline fused with Phenanthrene |
| A6_2A4_4N1Ph2BN2 | 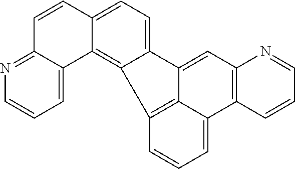 [SMILES: c1cnc2cc3c4c(cccc4c2c1)-c1c-3ccc2ccc3ncccc3c12] | Benzoquinoline fused with Benzoquinoline |
| A6_3A4_2N1Ph3BN1 | 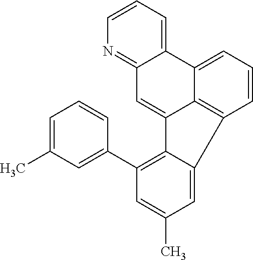 [SMILES: Cc1cccc(-c2cc(C)cc3c2-c2cc4ncccc4c4cccc-3c24)c1] | Biphenyl fused with Benzoquinoline |
| A6_3A4_1N1Ph3HT1 | 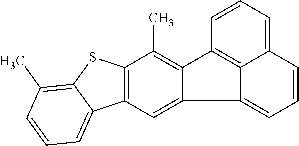 [SMILES: Cc1cccc2c1sc1c(C)c3c(cc12)-c1cccc2cccc-3c12] | Dibenzothiophene (hard sulfur removal) fused with naphthalene |
| A6_3A4_1N1Ph3HT1BN1 | 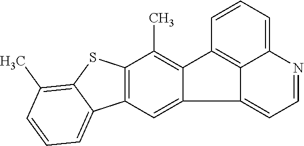 [SMILES: Cc1cccc2c1sc1c(C)c3c(cc12)-c1ccnc2cccc-3c12] | Dibenzothiophene (hard sulfur removal) fused with quinoline |
| A6_3N1Ph3HT2 | 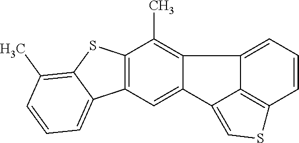 [SMILES: Cc1cccc2c1sc1c(C)c3c(cc12)-c1csc2cccc-3c12] | Dibenzothiophene (hard sulfur removal) fused with benzothiophene |
| A6_3N1Ph3HT1NN1 | 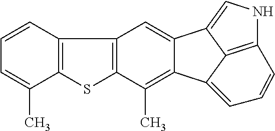 [SMILES: Cc1cccc2c1sc1c(C)c3c(cc12)-c1c[nH]c2cccc-3c12] | Dibenzothiophene (hard sulfur removal) fused with indole |
| A6_3A4_2N1Ph3HT1 | 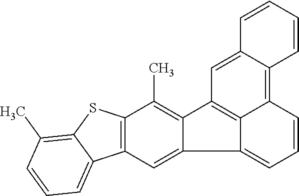 [SMILES: Cc1cccc2c1sc1c(C)c3c(cc12)-c1cccc2c1c-3cc1ccccc12] | Dibenzothiophene (hard sulfur removal) fused with phenanthrene |
| A6_3A4_2N1Ph3HT1BN1 | 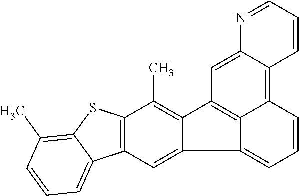 [SMILES: Cc1cccc2c1sc1c(C)c3c(cc12)-c1cccc2c1c-3cc1ncccc12] | Dibenzothiophene (hard sulfur removal) fused with Benzoquinoline |
| A6_3A4_1N1Ph3ET1 | 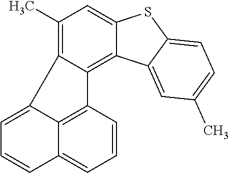 [SMILES: Cc1ccc2sc3cc(C)c4c(c3c2c1)-c1cccc2cccc-4c12] | Dibenzothiophene (not hard sulfur removal) fused with naphthalene |
| A6_3A4_1N1Ph3ET1BN1 | 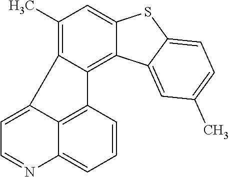 [SMILES: Cc1ccc2sc3cc(C)c4c(c3c2c1)-c1cccc2nccc-4c12] | Dibenzothiophene (not hard sulfur removal) fused with benzoquinoline |
| A6_3N1Ph3ET2 | 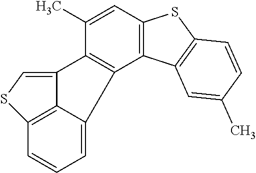 [SMILES: Cc1ccc2sc3cc(C)c4c(c3c2c1)-c1cccc2scc-4c12] | Dibenzothiophene (not hard sulfur removal) fused with benzothiophene |
| A6_3N1Ph3ET1NN1 | 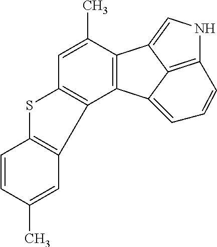 [SMILES: Cc1ccc2sc3cc(C)c4c(c3c2c1)-c1cccc2[nH]cc-4c12] | Dibenzothiophene (not hard sulfur removal) fused with indole |
| A6_3A4_2N1Ph3ET1 | 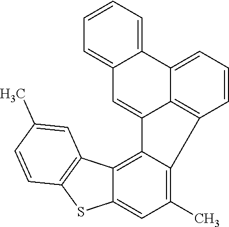 [SMILES: Cc1ccc2sc3cc(C)c4c(c3c2c1)-c1cc2ccccc2c2cccc-4c12] | Dibenzothiophene (not hard sulfur removal) fused with phenanthrene |
| A6_3A4_2N1Ph3ET1BN1 | 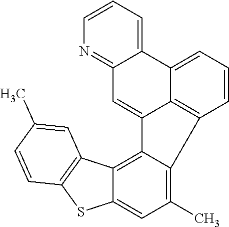 [SMILES: Cc1ccc2sc3cc(C)c4c(c3c2c1)-c1cc2ncccc2c2cccc-4c12] | Dibenzothiophene (not hard sulfur removal) fused with benzoquinoline |
| A6_3A4_1N1Ph3NN1 | 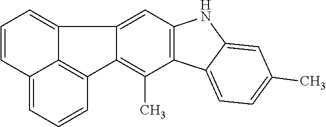 [SMILES: Cc1ccc2c(c1)[nH]c1cc3c(c(C)c12)-c1cccc2cccc-3c12] | Carbazole fused with naphthalene |
| A6_3A4_1N1Ph3BN1NN1 | 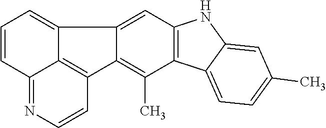 [SMILES: Cc1ccc2c(c1)[nH]c1cc3c(c(C)c12)-c1ccnc2cccc-3c12] | Carbazole fused with quinoline |
| A6_3N1Ph3NN2 | 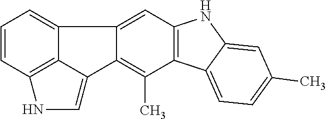 [SMILES: Cc1ccc2c(c1)[nH]c1cc3c(c(C)c12)-c1c[nH]c2cccc-3c12] | Carbazole fused with indole |
| A6_3A4_2N1Ph3NN1 | 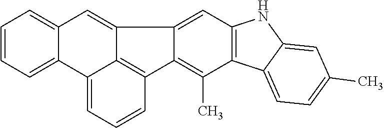 [SMILES: Cc1ccc2c(c1)[nH]c1cc3c(c(C)c12)-c1cccc2c1c-3cc1ccccc12] | Carbazole fused with phenanthrene |
| A6_3A4_2N1Ph3BN1NN1 | 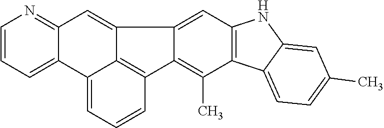 [SMILES: Cc1ccc2c(c1)[nH]c1cc3c(c(C)c12)-c1cccc2c1c-3cc1ncccc12] | Carbazole fused with benzoquinoline |
| A6_3A4_3N1Ph3 | 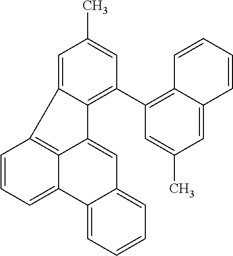 [SMILES: Cc1cc(-c2cc(C)cc3ccccc23)c2c(c1)-c1cccc3c1c-2cc1ccccc13] | Biphenyl fused with chrysene |
| A6_3A4_3N1Ph3BN1 | 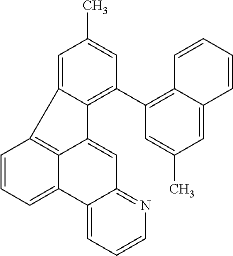 [SMILES: Cc1cc(-c2cc(C)cc3ccccc23)c2c(c1)-c1cccc3c1c-2cc1ncccc13] | Biphenyl fused with naphthoquinoline |
| A6_4A4_2N1Ph4 | 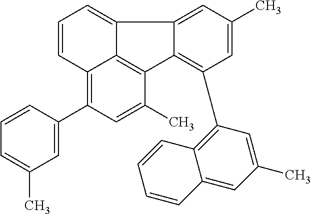 [SMILES: Cc1cccc(-c2cc(C)c3c4c(cccc24)-c2cc(C)cc(-c4cc(C)cc5ccccc45)c2-3)c1] | Phenylnaphthalene fused with phenylnaphthalene |
| A6_4A4_1N1Ph4 | 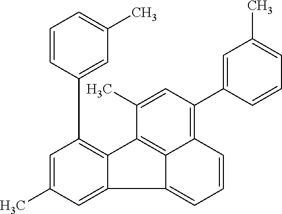 [SMILES: Cc1cccc(-c2cc(C)cc3c2-c2c(C)cc(-c4cccc(C)c4)c4cccc-3c24)c1] | Biphenyl fused with phenylnaphthalene |
| A6_4A4_1N1Ph4HT1 | 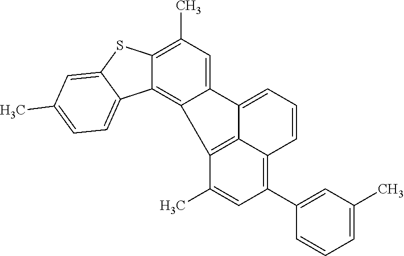 | Naphthobenzothiophene (hard sulfur removal) fused with phenylnaphthalene |
| A6_4A4_2N1Ph2 | 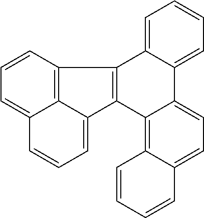 [SMILES: c1cc2c3c(cccc3c1)-c1c-2c2ccccc2c2ccc3ccccc3c12] | Chrysene fused with naphthalene |
| A6_4A4_2N1Ph2BN1 | 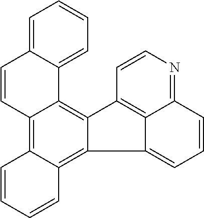 [SMILES: c1ccc2c(c1)ccc1c3ccccc3c3c(c21)-c1ccnc2cccc-3c12] | Chrysene fused with quinoline |
| A6_4A4_1N1Ph2ET1 | 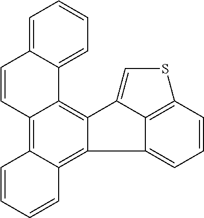 [SMILES: c1ccc2c(c1)ccc1c3ccccc3c3c(c21)-c1csc2cccc-3c12] | Chrysene fused with benzothiophene |
| A6_4A4_1N1Ph2NN1 | 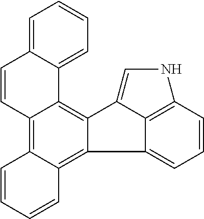 [SMILES: c1ccc2c(c1)ccc1c3ccccc3c3c(c21)-c1c[nH]c2cccc-3c12] | Chrysene fused with indole |
| A6_4A4_3N1Ph2 | 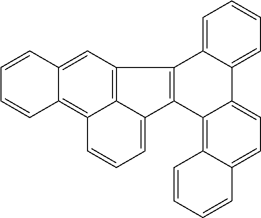 [SMILES: c1ccc2c(c1)cc1c3c(cccc32)-c2c-1c1ccccc1c1ccc3ccccc3c21] | Chrysene fused with phenanthrene |
| A6_4A4_3N1Ph2BN1 | 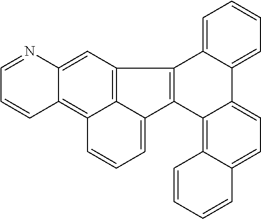 [SMILES: c1ccc2c(c1)ccc1c3ccccc3c3c(c21)-c1cccc2c1c-3cc1ncccc12] | Chrysene fused with benzoquinoline |
| A6_5A4_2N1Ph3 | 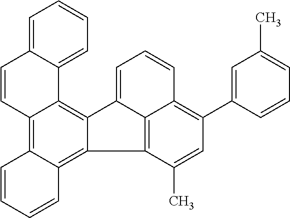 [SMILES: Cc1cccc(-c2cc(C)c3c4c(cccc24)-c2c-3c3ccccc3c3ccc4ccccc4c23)c1] | Chrysene fused with phenylnaphthalene |
| A6_2A4_3N1Ph2ET1 | 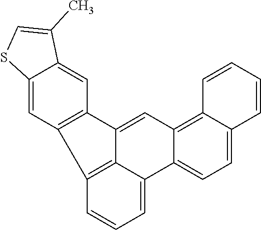 [SMILES: Cc1csc2cc3c(cc12)-c1cc2c4ccccc4ccc2c2cccc-3c12] | Chrysene fused with benzothiophene1 |
| A6_2A4_3N1Ph2NN1 | 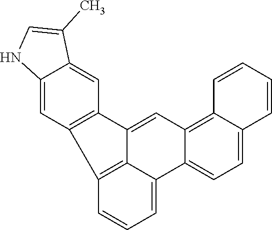 [SMILES: Cc1c[nH]c2cc3c(cc12)-c1cc2c4ccccc4ccc2c2cccc-3c12] | Chrysene fused with indole1 |
| A6_4A4_2N1Ph2BN2 | 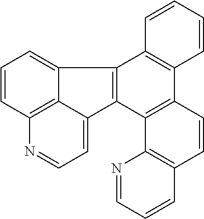 [SMILES: c1cnc2c(c1)ccc1c3ccccc3c3c(c12)-c1ccnc2cccc-3c12] | Naphthoquinoline fused with quinoline |
| A6_4A4_1N1Ph2ET1BN1 | 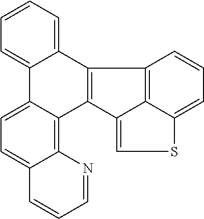 [SMILES: c1cnc2c(c1)ccc1c3ccccc3c3c(c12)-c1csc2cccc-3c12] | Naphthoquinoline fused with benzothiophene |
| A6_4A4_1N1Ph2BN1NN1 | 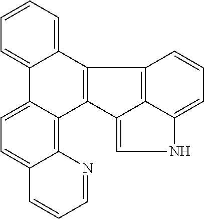 [SMILES: c1cnc2c(c1)ccc1c3ccccc3c3c(c12)-c1c[nH]c2cccc-3c12] | Naphthoquinoline fused with indole |
| A6_4A4_3N1Ph2BN2 | 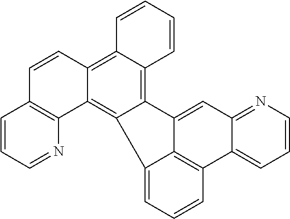 [SMILES: c1cnc2c(c1)ccc1c3ccccc3c3c(c12)-c1cccc2c1c-3cc1ncccc12] | Naphthoquinoline fused with benzoquinoline |
| A6_5A4_2N1Ph3BN1 | 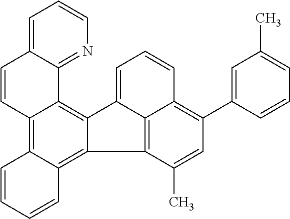 [SMILES: Cc1cccc(-c2cc(C)c3c4c(cccc24)-c2c-3c3ccccc3c3ccc4cccnc4c23)c1] | Naphthoquinoline fused with phenylnaphthalene |
| A6_2A4_3N1Ph2ET1BN1 | 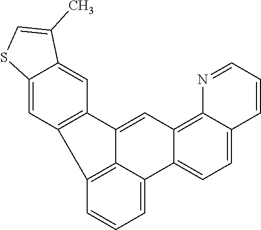 [SMILES: Cc1csc2cc3c(cc12)-c1cc2c(ccc4cccnc42)c2cccc-3c12] | Naphthoquinoline fused with benzothiophene1 |
| A6_2A4_3N1Ph2BN1NN1 | 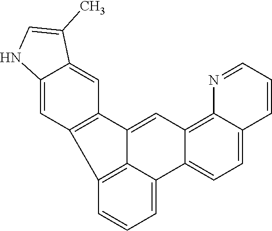 [SMILES: Cc1c[nH]c2cc3c(cc12)-c1cc2c(ccc4cccnc42)c2cccc-3c12] | Naphthoquinoline fused with indole1 |
| A6_3A4_1N1Ph3HT2 | 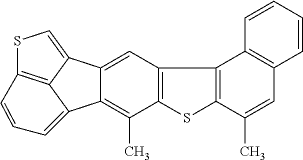 [SMILES: Cc1c2c(cc3c1sc1c(C)cc4ccccc4c13)-c1csc3cccc-2c13] | Naphthobenzothiophene (hard sulfur removal) fused with benzothiophene |
| A6_3A4_1N1Ph3HT1NN1 | 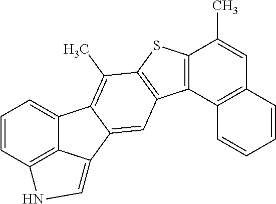 [SMILES: Cc1c2c(cc3c1sc1c(C)cc4ccccc4c13)-c1c[nH]c3cccc-2c13] | Naphthobenzothiophene (hard sulfur removal) fused with indole |
| A6_3A4_3N1Ph3HT1 | 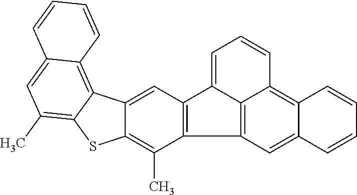 [SMILES: Cc1c2c(cc3c1sc1c(C)cc4ccccc4c13)-c1cccc3c1c-2cc1ccccc13] | Naphthobenzothiophene (hard sulfur removal) fused with naphthalene |
| A6_3A4_3N1Ph3HT1BN1 | 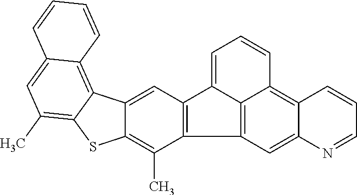 [SMILES: Cc1c2c(cc3c1sc1c(C)cc4ccccc4c13)-c1cccc3c1c-2cc1ncccc13] | Naphthobenzothiophene (hard sulfur removal) fused with benzoquinoline |
| A6_4A4_2N1Ph4HT1 | 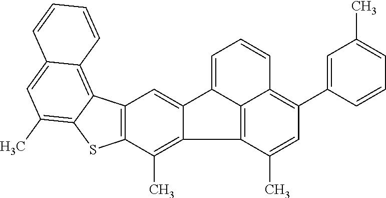 [SMILES: Cc1cccc(-c2cc(C)c3c4c(cccc24)-c2cc4c(sc5c(C)cc6ccccc6c54)c(C)c2-3)c1] | Naphthobenzothiophene (hard sulfur removal) fused with biphenyl |
| A6_3A4_1N1Ph3ET2 | 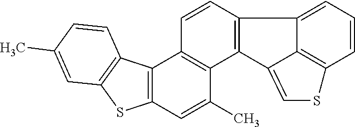 [SMILES: Cc1ccc2c(c1)sc1cc(C)c3c4c(ccc3c12)-c1cccc2scc-4c12] | Naphthobenzothiophene (not hard sulfur removal) fused with benzothiophene |
| A6_3A4_1N1Ph3ET1NN1 | 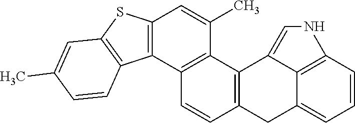 [SMILES: Cc1ccc2c(c1)sc1cc(C)c3c4c(ccc3c12)Cc1cccc2[nH]cc-4c12] | Naphthobenzothiophene (not hard sulfur removal) fused with indole |
| A6_3A4_3N1Ph3ET1 | 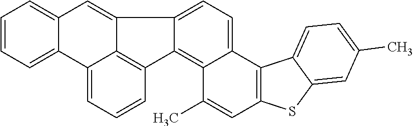 [SMILES: Cc1ccc2c(c1)sc1cc(C)c3c4c(ccc3c12)-c1cc2ccccc2c2cccc-4c12] | Naphthobenzothiophene (not hard sulfur removal) fused with naphthalene |
| A6_3A4_3N1Ph3ET1BN1 | 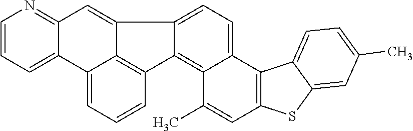 [SMILES: Cc1ccc2c(c1)sc1cc(C)c3c4c(ccc3c12)-c1cc2ncccc2c2cccc-4c12] | Naphthobenzothiophene (not hard sulfur removal) fused with benzoquinoline |
| A6_4A4_2N1Ph4ET1 | 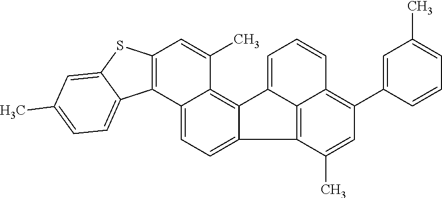 [SMILES: Cc1cccc(-c2cc(C)c3c4c(cccc24)-c2c-3ccc3c2c(C)cc2sc4cc(C)ccc4c23)c1] | Naphthobenzothiophene (not hard sulfur removal) fused with phenylnaphthalene |
| A6_3A4_1N1Ph3NN2 | 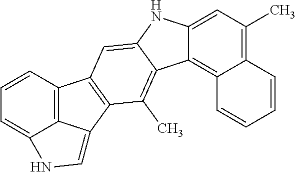 [SMILES: Cc1cc2[nH]c3cc4c(c(C)c3c2c2ccccc12)-c1c[nH]c2cccc-4c12] | Benzocarbazole (not hard sulfur removal) fused with indole |
| A6_3A4_3N1Ph3NN1 | 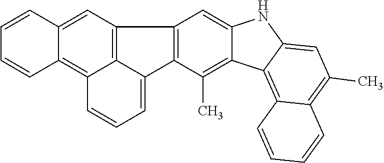 [SMILES: Cc1cc2[nH]c3cc4c(c(C)c3c2c2ccccc12)-c1cccc2c1c-4cc1ccccc12] | Benzocarbazole (not hard sulfur removal) fused with naphthalene |
| A6_3A4_3N1Ph3BN1NN1 | 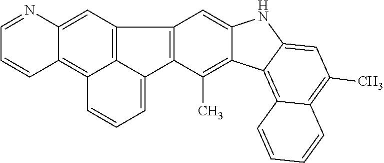 [SMILES: Cc1cc2[nH]c3cc4c(c(C)c3c2c2ccccc12)-c1cccc2c1c-4cc1ncccc12] | Benzocarbazole (not hard sulfur removal) fused with quinoline |
| A6_4A4_2N1Ph4NN1 | 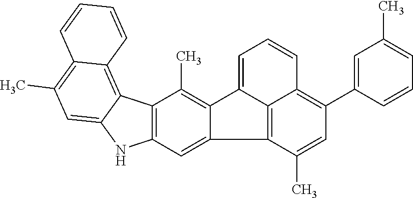 [SMILES: Cc1cccc(-c2cc(C)c3c4c(cccc24)-c2c-3cc3[nH]c4cc(C)c5ccccc5c4c3c2C)c1] | Benzocarbazole (not hard sulfur removal) fused with phenylnaphthalene |
| A6_2A4_5N1Ph2 | 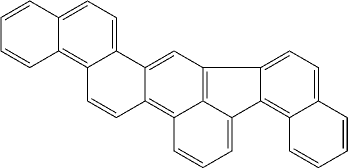 [SMILES: c1ccc2c3c(ccc2c1)-c1cc2c(ccc4c5ccccc5ccc42)c2cccc-3c12] | Picene fused with naphthalene |
| A6_2A4_5N1Ph2BN1 | 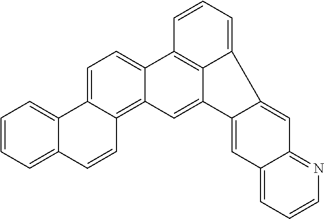 [SMILES: c1cnc2cc3c(cc2c1)-c1cc2c(ccc4c5ccccc5ccc42)c2cccc-3c12] | Picene fused with quinoline |
| A6_3A4_4N1Ph3 | 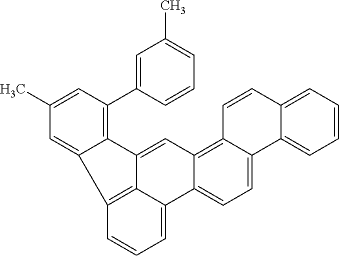 [SMILES: Cc1cccc(-c2cc(C)cc3c2-c2cc4c(ccc5c6ccccc6ccc54)c4cccc-3c24)c1] | Picene fused with biphenyl |
| A6_4A4_1N1Ph4ET1 | 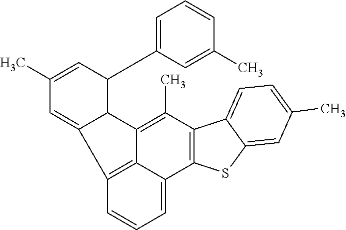 | Naphthobenzothiophene (not hard sulfur removal) fused with biphenyl |
| A6_4A4_2N1Ph2ET1 | 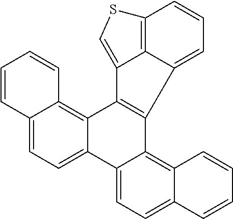 [SMILES: c1ccc2c(c1)ccc1c3ccc4ccccc4c3c3c(c21)-c1cccc2scc-3c12] | Picene fused with benzothiophene |
| A6_4A4_2N1Ph2NN1 | 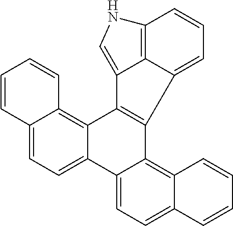 [SMILES: c1ccc2c(c1)ccc1c3ccc4ccccc4c3c3c(c21)-c1cccc2[nH]cc-3c12] | Picene fused with indole |
| A6_4A4_4N1Ph2 | 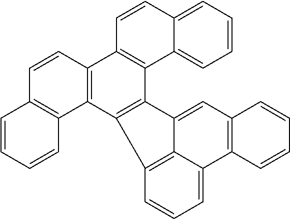 [SMILES: c1ccc2c(c1)cc1c3c(cccc32)-c2c-1c1c3ccccc3ccc1c1ccc3ccccc3c21] | Picene fused with phenanthrene |
| A6_4A4_4N1Ph2BN1 | 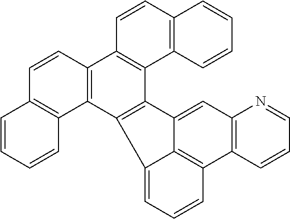 [SMILES: c1ccc2c(c1)ccc1c3ccc4ccccc4c3c3c(c21)-c1cccc2c1c-3cc1ncccc12] | Picene fused with benzoquinoline |
| A6_5A4_3N1Ph3 | 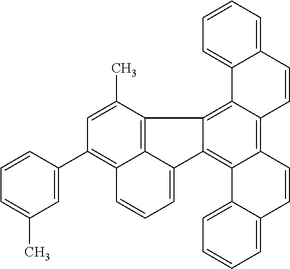 [SMILES: Cc1cccc(-c2cc(C)c3c4c(cccc24)-c2c-3c3c4ccccc4ccc3c3ccc4ccccc4c23)c1] | Picene fused with phenylnaphthalene |
| A6_2A4_4N1Ph2ET1 | 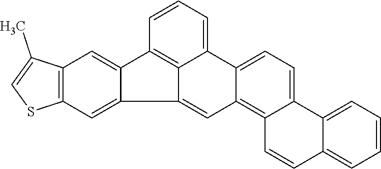 [SMILES: Cc1csc2cc3c(cc12)-c1cccc2c1c-3cc1c2ccc2c3ccccc3ccc21] | Picene fused with benzothiophene1 |
| A6_2A4_4N1Ph2NN1 | 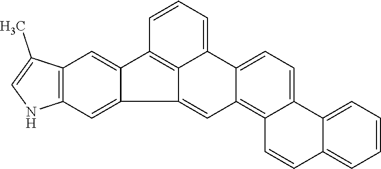 [SMILES: Cc1c[nH]c2cc3c(cc12)-c1cccc2c1c-3cc1c2ccc2c3ccccc3ccc21] | Picene fused with indole1 |
| A6_2A4_6N1Ph2 | 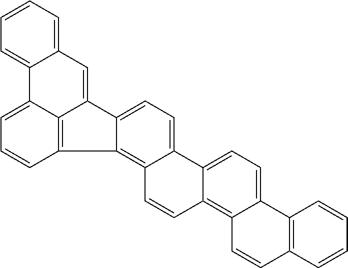 [SMILES: c1ccc2c(c1)cc1c3c(cccc32)-c2c-1ccc1c2ccc2c1ccc1c3ccccc3ccc12] | Picene fused with phenanthrene1 |
| A6_2A4_6N1Ph2BN1 | 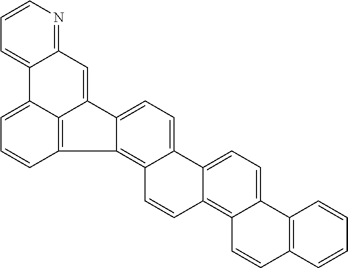 [SMILES: c1ccc2c(c1)ccc1c2ccc2c3ccc4c(c3ccc21)-c1cccc2c1c-4cc1ncccc12] | Picene fused with benzoquinoline1 |
| A6_3A4_5N1Ph3 | 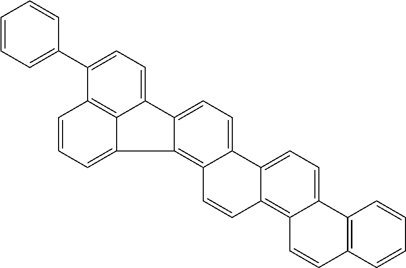 [SMILES: c1ccc(-c2ccc3c4c(cccc24)-c2c-3ccc3c2ccc2c3ccc3c4ccccc4ccc32)cc1] | Picene fused with phenylnaphthalene1 |

In general, including a greater number of molecular types improves resolution and/or accuracy, but requires additional computing resources. It is not necessary to include all of the molecular types identified in Tables 1.1 and 1.2, and selected subsets can be used. For example, based on known information regarding a particular set of chemical reactions (e.g., analytical data regarding the composition of a hydrocarbon mixture), some molecular types can be omitted. Other molecular types can be omitted in view of anticipated reactor conditions (e.g., temperature and pressure), which can influence the reactions that are likely to occur within the reactor.

In general, in embodiments there is no required molecular type or minimum number of molecular types. The particular molecule types used and the total number of molecule types can depend on the feedstock and chemistries. For example, if modeling paraffin hydroisomerization, only normal paraffin, isoparaffin with one branch, and isoparaffin with multiple branches can be necessary. If modeling naphtha hydrotreating, molecular types having two or more than two rings are not required. Selection of the appropriate molecular types is dependent upon the feedstock and reaction chemistry.

### Reactions

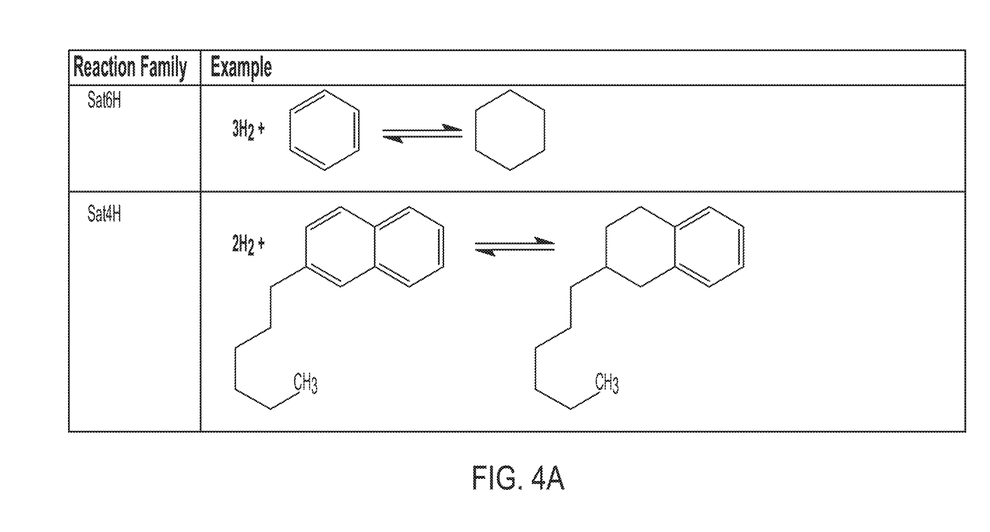

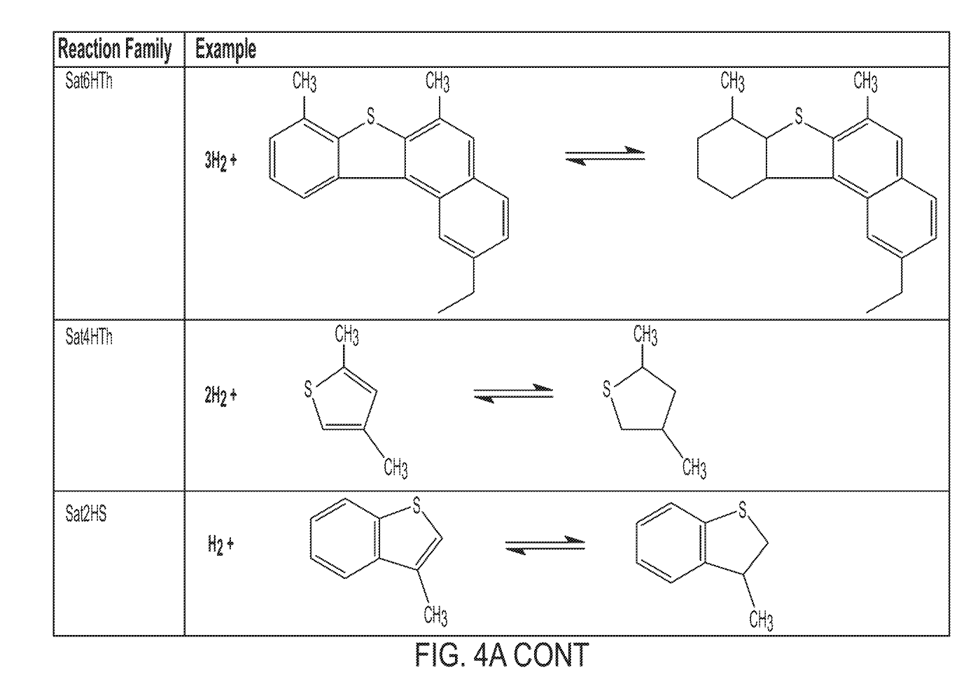

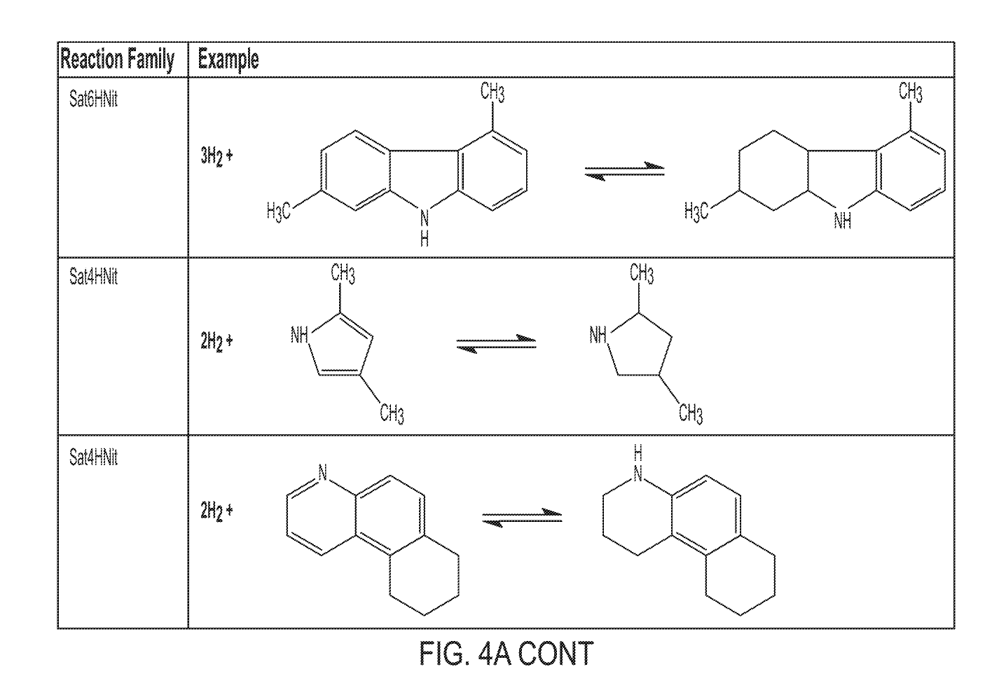

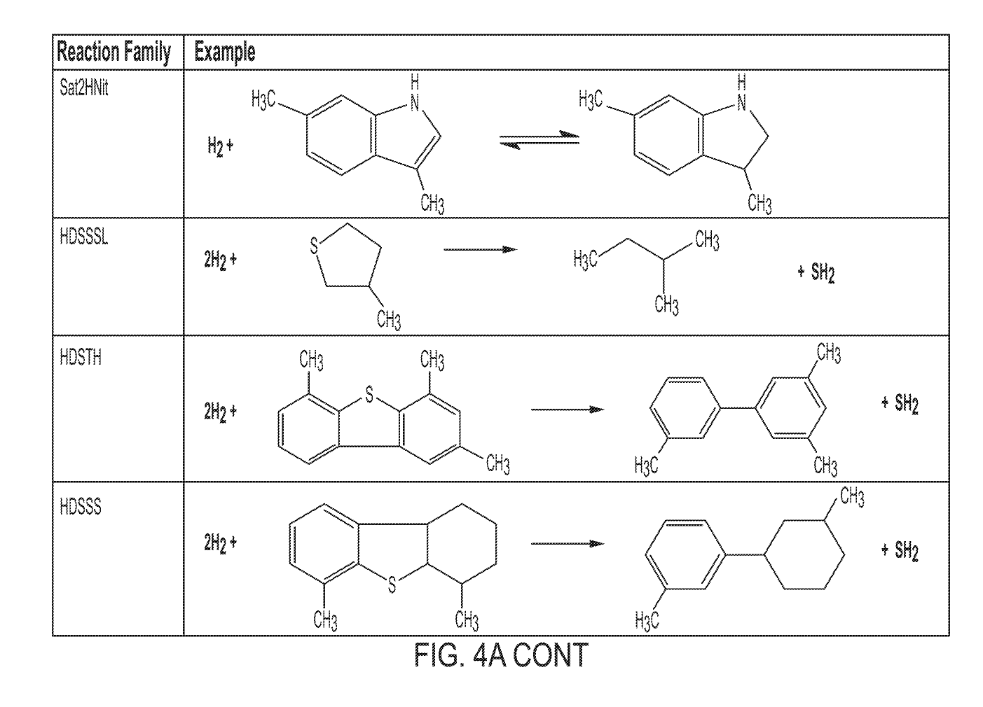

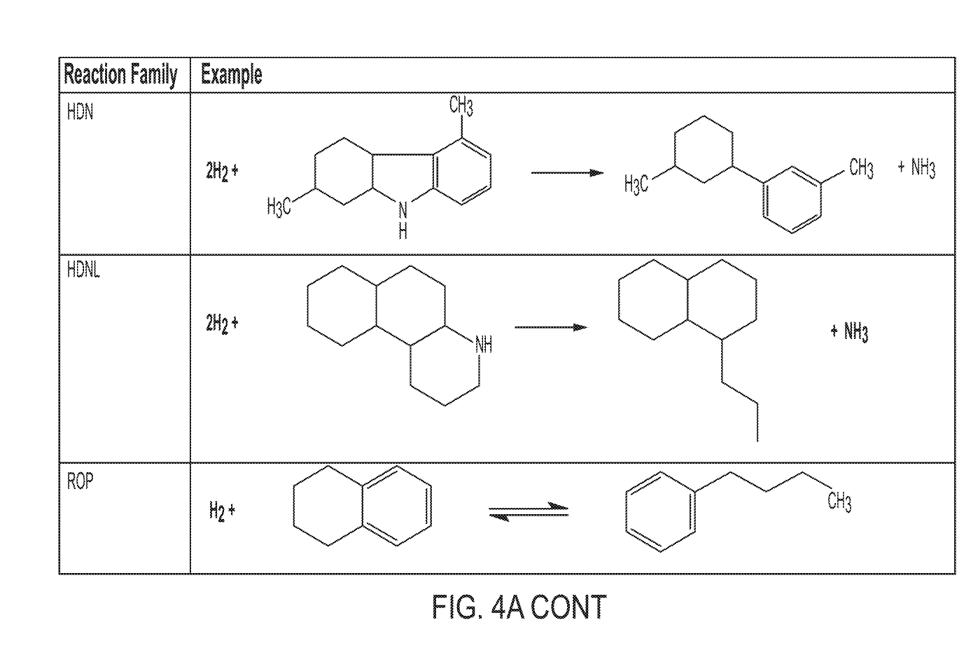

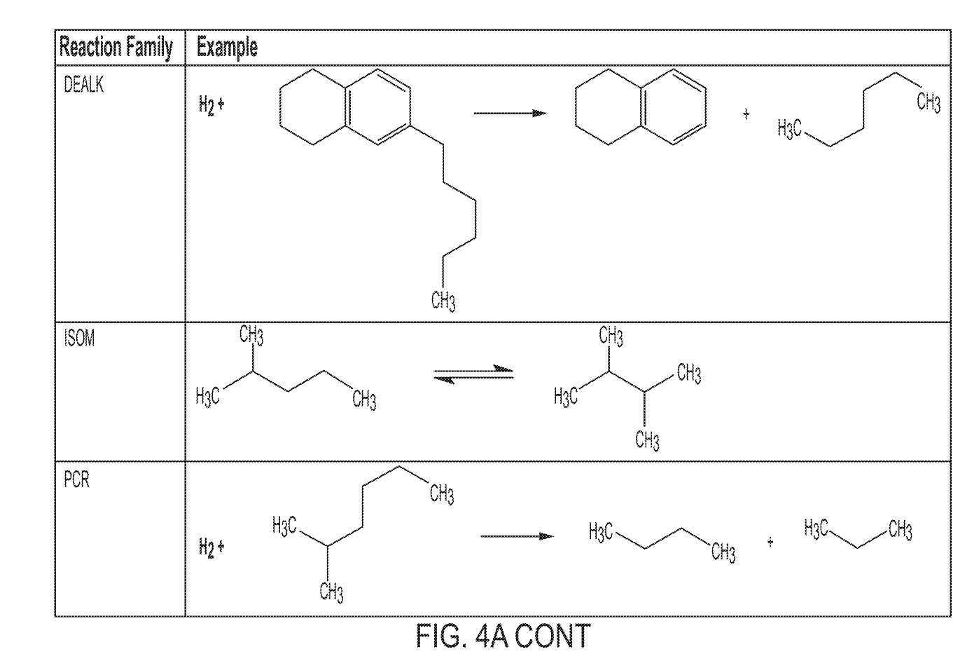

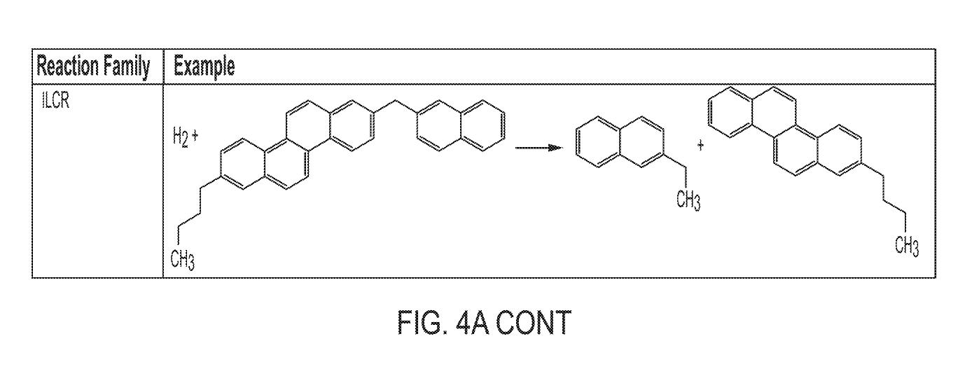

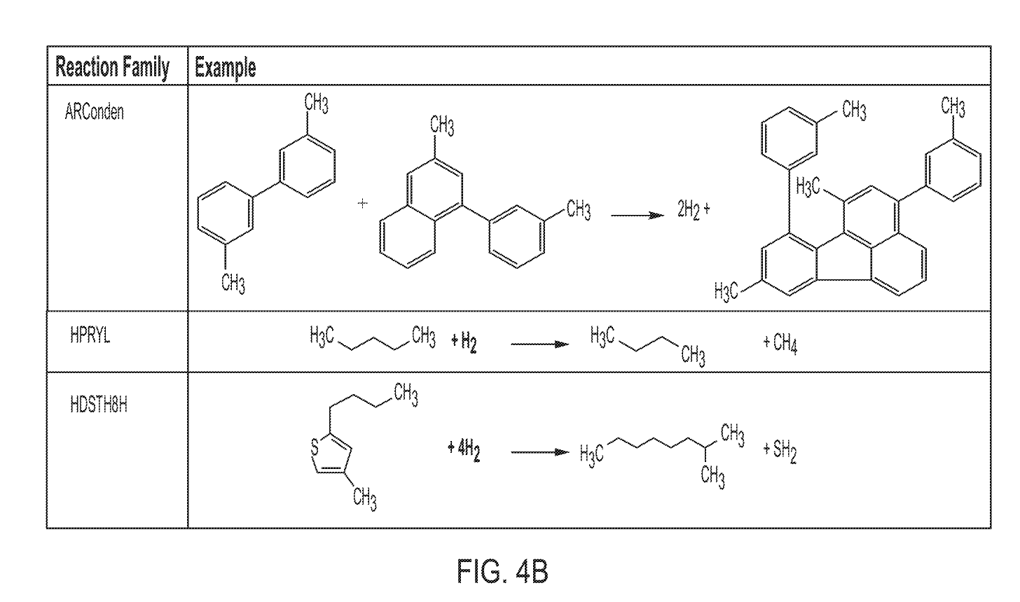

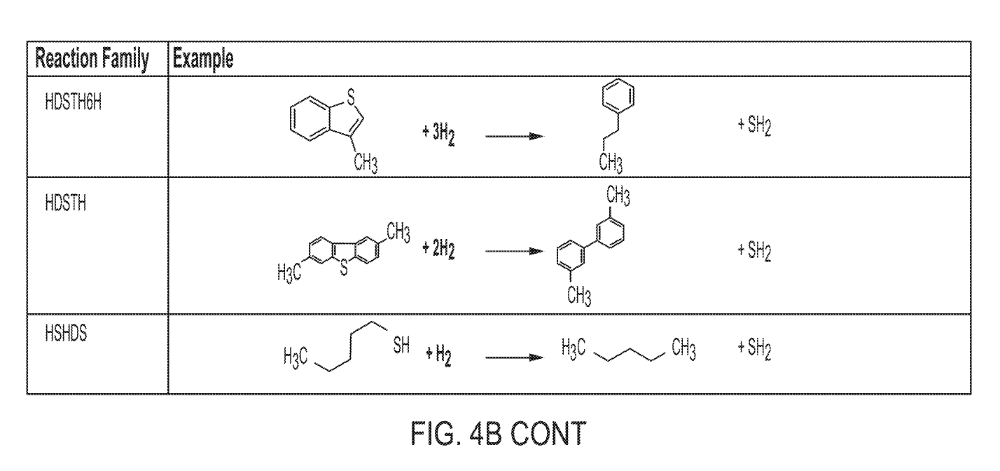

FIGS. 4A and 4B are tables that depict examples of permissible reactions that can occur within a reactor considered by reaction network 125. In some embodiments, the permissible reactions are grouped according to reaction family. For example, the Saturation6H reaction family (shown at the first row in FIG. 4A) pertains to saturation reactions of a benzene ring in a pure hydrocarbon with 3H₂. The particular reaction identified is a reversible reaction in which a benzene ring reacts with 3H₂ (a total of six hydrogen atoms) to form a saturated cyclohexane ring. The Saturation 4H reaction family (shown at the second row of FIG. 4A) pertains to saturation reactions of a benzene ring in a pure hydrocarbon with 2H₂. The particular reaction identified involves a reaction of an alkyl naphthalene with 2H₂. The ring opening (ROP) reaction family pertains to reactions in which a naphthenic ring is opened. In multicore structures, such as A6_3A4_2Ph1HT1 (phenanthrene connected with dibenzothiophene), the interlink between the cores is typically cracked prior to desulfurization reactions.

Tables 2.1, 2.2 and 2.3 are non-exclusive lists of reaction families, associated descriptions, and number of reactions utilized by reaction network 125. As an example, applying a set of permissible hydrocracking reactions to a set of reactor compounds yields 1366 species and 3186 reactions, as indicated in Table 2.1.

TABLE 2.1
Reaction families and associated statistics for a hydrocracker (HCR) reaction network
125 for Example 1.

| Reaction Family | | |
|---|---|---|
| (permissible | | |
| reactions) | Description of Reaction Family | # of Reactions |
| Sat6HTh | Saturate a benzene ring in thiophenics with 3 H₂ | 144 |
| Sat6HNit | Saturate a benzene ring in pyridinics or pyrrolics | 101 |
| | with 3 H₂ | |
| Sat6H | Saturate a benzene ring in pure hydrocarbon | 143 |
| | with 3 H₂ | |
| Sat4HTh | Saturate an isolated thiophenic ring with 2 H₂ | 34 |
| Sat4HNit | Saturate a pyridinic ring fused with a benzene | 112 |
| | ring with 2 H₂; or saturate an isolated pyrrolic | |
| | ring with 2 H₂ | |
| Sat4H | Saturate a benzene ring in pure hydrocarbon | 242 |
| | with 2 H₂ | |
| Sat2HS | Saturate a thiophenic ring fused with a benzene | 27 |
| | ring with 1 H₂ | |
| Sat2HNit | Saturate a pyrrolic ring fused with a benzene | 28 |
| | ring with 1 H₂ | |
| ROP | Naphthenics ring opening | 108 |
| PCR | Paraffin hydrocracking | 128 |
| ISOM | Paraffin isomerization | 57 |
| HDSTH | Desulfurization of dibenzothiophene structures | 94 |
| HDSSS | Desulfurization of saturated thiophenics in | 91 |
| | saturated dibenzothiophene structures | |
| HDSSSL | Desulfurization of saturated benzothiophene; or | 58 |
| | desulfurization of saturated thiophene structures | |
| HDN | Denitrogenation of saturated nitrogen rings in | 106 |
| | saturated carbazole structures | |
| HDNL | Denitrogenation of saturated nitrogen rings in | 133 |
| | saturated indoles, pyrroles, pyridine, or quinine | |
| | structures | |
| DEALK | Dealkylation | 1580 |
| Total | | 3186 |

TABLE 2.2
Reaction families and associated statistics for a hydrocracker (HCR) reaction network
125 for Example 2.

| Reaction Family | | |
|---|---|---|
| (permissible | | |
| reactions) | Description of Reaction Family | # of Reactions |
| Sat6HTh | Saturate a benzene ring in thiophenics with 3 H₂ | 96 |
| Sat6HNit | Saturate a benzene ring in pyridinics or pyrrolics | 101 |
| | with 3 H₂ | |
| Sat6H | Saturate a benzene ring in pure hydrocarbon | 418 |
| | with 3 H₂ | |
| Sat4HTh | Saturate an isolated thiophenic ring with 2 H₂ | 34 |
| Sat4HNit | Saturate a pyridinic ring fused with a benzene | 156 |
| | ring with 2 H₂; or saturate an isolated pyrrolic | |
| | ring with 2 H₂ | |
| Sat4H | Saturate a benzene ring in pure hydrocarbon | 329 |
| | with 2 H₂ | |
| Sat2HS | Saturate a thiophenic ring fused with a benzene | 27 |
| | ring with 1 H₂ | |
| Sat2HNit | Saturate a pyrrolic ring fused with a benzene | 28 |
| | ring with 1 H₂ | |
| ROP | Naphthenics ring opening | 382 |
| PCR | Paraffin hydrocracking | 162 |
| ISOM | Paraffin isomerization | 55 |
| HPRYL | Paraffin hydrogenolysis | 6 |
| HDSTH | Desulfurization of dibenzothiophene structures | 94 |
| HDSSS | Desulfurization of saturated thiophenics in | 149 |
| | saturated dibenzothiophene structures | |
| HDSTH6H | Desulfurization of benzothiophene structures | 31 |
| HDN | Denitrogenation of saturated nitrogen rings in | 279 |
| | saturated indoles, pyrroles, pyridine, carbazole, | |
| | or quinine structures | |
| DEALK | Dealkylation | 2720 |
| HSHDS | Desulfurization of sulfides | 38 |
| ILCR | Inter-core linkage cracking of resid molecules | 595 |
| HDSTH8H | Desulfurization of thiophene structures | 34 |
| Sum | | 5734 |

TABLE 2.3
Reaction families and associated statistics for a hydrocracker (HCR) reaction network
125 for Example 3.

| Reaction Family | | |
|---|---|---|
| (permissible | | |
| reactions) | Description of Reaction Family | # of Reactions |
| Sat6HTh | Saturate a benzene ring in thiophenics with 3 H₂ | 96 |
| Sat6HNit | Saturate a benzene ring in pyridinics or pyrrolics | 81 |
| | with 3 H₂ | |
| Sat6H | Saturate a benzene ring in pure hydrocarbon | 369 |
| | with 3 H₂ | |
| Sat4HTh | Saturate an isolated thiophenic ring with 2 H₂ | 34 |
| Sat4HNit | Saturate a pyridinic ring fused with a benzene | 133 |
| | ring with 2 H₂; or saturate an isolated pyrrolic | |
| | ring with 2 H₂ | |
| Sat4H | Saturate a benzene ring in pure hydrocarbon | 329 |
| | with 2 H₂ | |
| Sat2HS | Saturate a thiophenic ring fused with a benzene | 27 |
| | ring with 1 H₂ | |
| Sat2HNit | Saturate a pyrrolic ring fused with a benzene | 28 |
| | ring with 1 H₂ | |
| ROP | Naphthenics ring opening | 328 |
| PCR | Paraffin hydrocracking | 162 |
| ISOM | Paraffin isomerization | 55 |
| HPRYL | Paraffin hydrogenolysis | 6 |
| HDSTH | Desulfurization of dibenzothiophene structures | 94 |
| HDSSS | Desulfurization of saturated thiophenics in | 149 |
| | saturated dibenzothiophene structures | |
| HDSTH6H | Desulfurization of benzothiophene structures | 31 |
| HDN | Denitrogenation of saturated nitrogen rings in | 279 |
| | saturated indoles, pyrroles, pyridine, carbazole, | |
| | or quinine structures | |
| DEALK | Dealkylation | 2521 |
| HSHDS | Desulfurization of sulfides | 38 |
| ILCR | Inter-core linkage cracking of resid molecules | 595 |
| HDSTH8H | Desulfurization of thiophene structures | 35 |
| ARConden | Aromatic Ring Condensation | 435 |
| Sum | | 5825 |

The Reaction Family HDN in Tables 2.2 and 2.3 includes additional reactions relative to Table 2.1, as indicated by the Description of Reaction Families in Tables 2.1, 2.2, and 2.3.

In general, including a greater number of reaction families (permissible reactions) improves resolution and/or accuracy, but requires additional computing resources. It is not necessary to include all of the reaction families (permissible reactions) identified in Tables 2.1, 2.2 and 2.3, and selected subsets can be used. For example, based on known information regarding a particular set of chemical reactions (e.g., analytical data regarding the composition of a hydrocarbon mixture), some reaction families (permissible reactions) can be omitted. Other reaction families (permissible reactions) can be omitted in view of anticipated reactor conditions (e.g., temperature and pressure), which can influence the reactions that are likely to occur within the reactor.

In general, there is no required reaction types or minimum number of reaction types in embodiments. The particular reaction types and total number of reactions in reaction network 125 can depend on the feedstock and chemistries. For example, modeling paraffin hydroisomerization, only the isomerization reaction family is necessary. Selection of the appropriate permissible reactions is dependent upon the feedstock and reaction chemistry.

### Properties of Reactor Compounds

The properties of the reactor compounds are a set of selected thermodynamic properties and physical properties. An example of a set of thermodynamic properties and physical properties of module 121 are shown in Table 3. A comprehensive thermodynamics model is not required in MB EORXR Block 155, and thus it is possible to exclude or omit some of the properties of Table 3. One of skill in the art will appreciate that including additional properties is likely to improve accuracy, but the tradeoff is an increased requirement for computing resources. Balancing accuracy and performance are tradeoffs that are well-understood in the art.

TABLE 3
Molecular properties used in Molecule-Based Equation
Oriented Reactor Block 155.

| Field Name | |
|---|---|
| of Property | Description of Property |
| MW | Molecular weight |
| CarbonNum | Total number of carbon atoms |
| HydrogenNum | Total number of hydrogen atoms |
| SideChainNum | Total number of side chains |
| AromRingNum | Total number of aromatic rings |
| NaphRingNum | Total number of naphthenic rings |
| ThphRingNum | Total number of thiophenic rings |
| PyrrolicRingNum | Total number of pyrrolic rings |
| PyridenicRingNum | Total number of pyridenic rings |
| TotalSulfur | Total number of sulfur atoms |
| TotalNitrogen | Total number of nitrogen atoms |
| TotalOxygen | Total number of oxygen atoms |
| AromCarbonNum | Total number of aromatic carbon atoms |
| NaphCarbonNum | Total number of naphthenic carbon atoms |
| ParCarbonNum | Total number of paraffinic carbon atoms |
| Naph6Ring | Total number of naphthenic six-carbon rings |
| Naph5Ring | Total number of naphthenic five-carbon rings |
| Tb | Boiling point |
| Density | Density |
| Hform | Standard enthalpy of formation in gas phase |
| Gform | Standard Gibbs free energy of formation in gas |
| | phase |
| Cp_a | Gas phase heat capacity coefficient a |
| Cp_b | Gas phase heat capacity coefficient b |
| Cp_c | Gas phase heat capacity coefficient c |
| Cp_d | Gas phase heat capacity coefficient d |
| HVap | Heat of vaporization |
| HfLiq | Standard enthalpy of formation in liquid phase |
| CpLiq_a | Liquid phase heat capacity coefficient a |
| CpLiq_b | Liquid phase heat capacity coefficient b |
| CpLiq_c | Liquid phase heat capacity coefficient c |
| ViscA | Coefficient A for viscosity |
| ViscB | Coefficient B for viscosity |
| ViscC | Coefficient C for viscosity |
| ViscD | Coefficient D for viscosity |
| Core | Molecular Type |

The heat capacity of gas phase is a 3^{rd} order polynomial:

Cp(T)=Cp_a+Cp_b·T+Cp_c·T²+Cp_d·T³ Eq. 3

The heat capacity of liquid phase is a 2^{nd} order polynomial:

Cp(T)=Cp_a+Cp_b·T+Cp_c·T² Eq. 4

The viscosity can be calculated from the following equation:

ln ηᵢ=Visc_Aᵢ+Visc_Bᵢ/(T+Visc_Dᵢ)+Visc_Cᵢ·ln T Eq. 5

MB Reactor Builder 150 may use Equations 3 through 5 among other component properties input from module 121 to generate the set of reaction rate equations 152 of a subject MB reactor model.

In general, including a greater number of properties of the reactor compounds improves resolution and/or accuracy, but requires additional computing resources. It is not necessary to include all of the properties of the reactor compounds identified in Table 3, and selected subsets can be used by MB reactor builder 150 in embodiments. For example, based on known information regarding a particular set of chemical reactions (e.g., analytical data regarding the composition of a hydrocarbon mixture), some properties of reactor compounds can be omitted. Other properties of reactor compounds can be omitted in view of anticipated reactor conditions (e.g., temperature and pressure), which can influence the reactions that are likely to occur within the reactor.

Typically, the properties of reactor compounds that are included as input from module 121 to MB reactor builder 150 are: total number of carbon atoms, total number of hydrogen atoms, total number of aromatic rings, total number of naphthenic rings, total number of thiophenic rings, total number of pyrrolic rings, total number of pyridenic rings, total number of sulfur atoms, total number of nitrogen atoms, total number of oxygen atoms, standard Gibbs free energy of formation, and standard enthalpy of formation. The remaining properties of reactor compounds are optional. If modeling a reduced subset of reactants (e.g., hydroisomerization), then some of reactor compounds that are ordinarily included would not be required. For example, for modeling hydroisomerization, the properties of reactor compounds that are typically included are total number of carbon atoms, total number of hydrogen atoms, standard Gibbs free energy of formation, and standard enthalpy of formation. Selection of the appropriate properties of reactor compounds is dependent upon the feedstock and reaction chemistry.

### Locally-Stored Pre-Estimated Thermodynamic Table

The properties of the molecular compositions in Molecule-Based Equation Oriented Reactor Block 155 are constant values. Thus, MB reactor builder 150 creates a local property table 151 to load into Molecule-Based Equation Oriented Reactor Block 155. Shown in Table 4 is one such local property (thermodynamic properties) table 151 generated and locally stored by MB reactor builder 150 in an embodiment. The data for the local property table 151 is pre-estimated, which reduces memory usage and improves speed in subsequent calculations. As a result, the number of species and reactions that can be handled overcomes the limitation of conventional equation-oriented models. Pre-estimation can be based on the “core structures.”

TABLE 4
Local thermodynamics properties 151 used in Molecule-Based Equation
Oriented Reactor Block 155.

| Field Name | |
|---|---|
| of Property | Description of Property |
| Hform(T) | Enthalpy of formation in gas phase at given temperature |
| Gform(T) | Gibbs free energy of formation in gas phase at given |
| | temperature |
| Cp(T) | Gas phase heat capacity at given temperature |
| S(T) | Entropy in gas phase at given temperature |
| HVap(T) | Heat of vaporization |
| HfLiq(T) | Enthalpy of formation in liquid phase at given temperature |
| CpLiq(T) | Liquid phase heat capacity at given temperature |

In general, including a greater number of pre-estimated thermodynamic properties 151 improves resolution and/or accuracy, but requires additional computing resources. It is not necessary to include all of the pre-estimated thermodynamic properties identified in Table 4, and selected subsets can be used. For example, based on known information regarding a particular set of chemical reactions (e.g., analytical data regarding the composition of a hydrocarbon mixture), some pre-estimated thermodynamic properties can be omitted. Other pre-estimated thermodynamic properties can be omitted in view of anticipated reactor conditions (e.g., temperature and pressure), which can influence the reactions that are likely to occur within the reactor.

In general, the pre-estimated thermodynamic properties 151 of Table 4 are for a given temperature, and it is used for non-isothermal conditions. The properties of the liquid phase are not used in the gas phase reactions and the properties of the gas phase are not used in the liquid phase. Mixed-phased reactions typically include the properties for both phases. Selection of the appropriate molecular types is dependent upon the feedstock and reaction chemistry.

Typically, the EO solver 175 utilizes three kinds of balance equations for a reactor model: mass balance equations, energy balance equations, and momentum balance equations.

### Coding Reaction Rate Equations

In many embodiments, the method 100 (at 152 in FIG. 1) includes automatically coding a set of reaction rate equations in equation-oriented (EO) format. The set of reaction rate equations 152 are typically coded based on the module 123 defined set of homologous series of compounds, the reaction network 125 defined set of permissible reactions, the module 121 defined properties of the reactor compounds, and the locally-stored pre-estimated thermodynamic properties 151.

For example, the set of rate equations 152 are Langmuir-Hinshelwood-Hougen-Watson (LHHW) rate laws. In addition, the kinetic parameters in LHHW are constrained by linear free energy relationship (LFER), which contributes to reducing the number of rate constant parameters. The set of reaction rate equations 152 includes one or more of residuals, sparsity patterns, and analytical Jacobians in equation-oriented format.

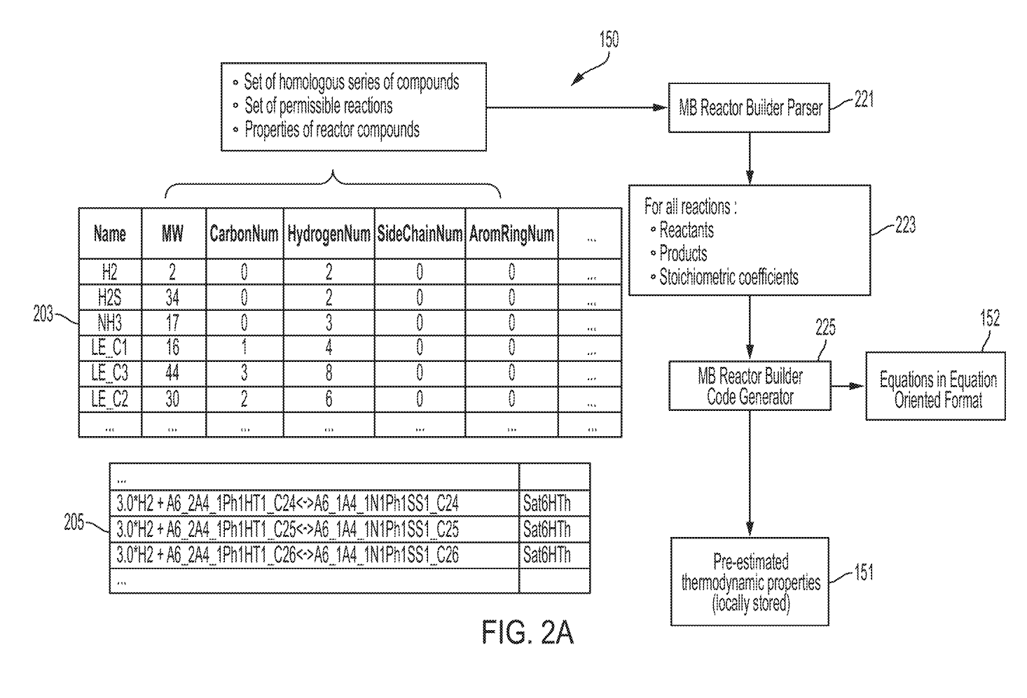

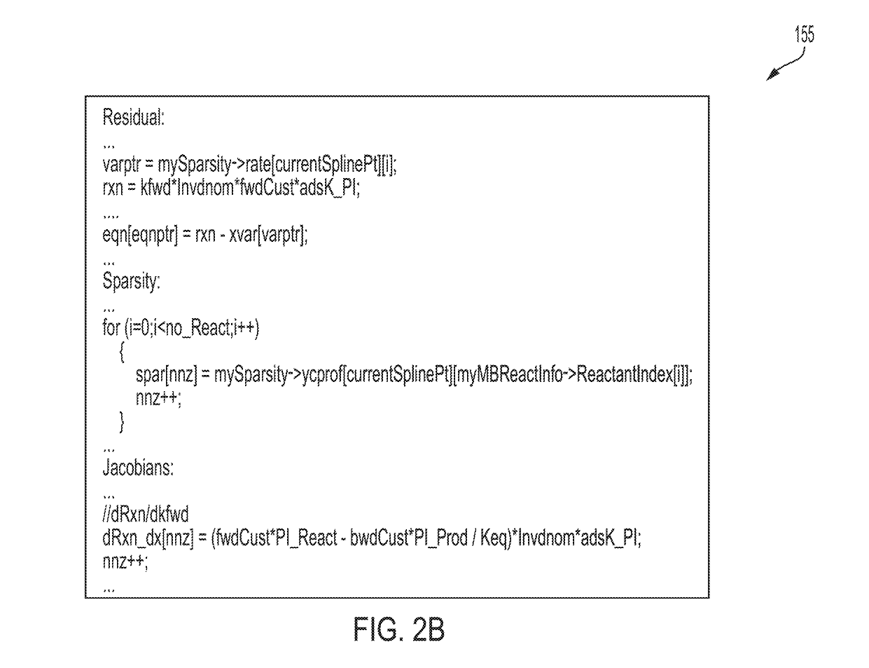

As shown in FIGS. 2A-B, the MB reactor builder 150 uses the defined set of homologous series of compounds from module 123 (e.g., Tables 1.1 and 1.2 and FIG. 3), defined set of permissible reactions from reaction network 125 (e.g., Tables 2.1, 2.2 and 2.3 and FIGS. 4A-B) and the defined properties of reactor compounds from module 121 (e.g., Table 3) as input data 203, 205. The MB reactor builder 150 has a parser 221 that parses the properties of the defined (input) series of homologous compounds 203 and that creates code to store and call the local thermodynamics property table 151. At step 223, the MB reactor builder 150/parser 221 parses the defined (input) set of permissible reactions 205 to obtain the reactants, products, and associated stoichiometry coefficients for the reactants and products. For each reaction, a code generator 225 of MB reactor builder 150 generates the code for Eq. 6 to Eq. 8 (e.g., set of reaction rate equations 152). In some embodiments, code generator 225 of MB reactor builder 150 generates the code for Eq. 6 to 10 (e.g., set of reaction rate equations 152). In some embodiments, code generator 225 of MB reactor builder 150 generates the code for Eq. 6 to 12 (e.g., set of reaction rate equations 152). The generated code (set of reaction rate equations 152) includes the residuals, sparsity patterns and analytical Jacobians in terms of EO format as illustrated in FIG. 2B and held as the EO formatted MB reactor model at MB EORXR block 155.

### Reduced Model

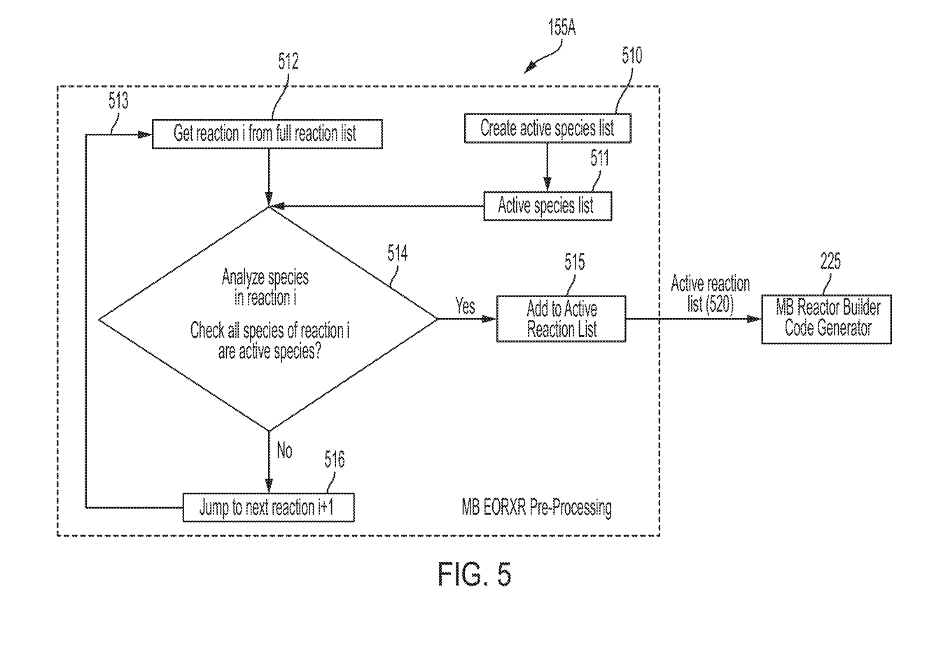

The initial reactor model generated by MB reactor builder 150 is referred to as a full reactor model. Additional benefits, particularly reduced processing time, can be achieved by creating a reduced reactor model from the full reactor model as illustrated in a non-limiting example of Reducer 150A in FIG. 5. As an overview, Reducer 155A operates subsequent to MB reactor builder 150 described above in FIGS. 1, 2A, and 2B and prior to MB EORXR Block 155. While the Reducer 155A is indicated to occur within MB EORXR Block 155, it could also occur within MB Reactor Builder 150 or as a separate component altogether.

A list of active species of the set of homologous series of compounds of module 123 is created 510 and stored 511. In other words, the list of active species is a reduced list 510, 511 compared to the full list 203 of species. By reducing the species list 203 to a list of active species, the computation demands are further reduced, which in turn can lead to a reduction in processing time.

For each permissible reaction from the full file 205 of reaction network 125, loop 513 analyzes the species (compound) in the subject reactor and determines 514 whether all the species in the subject reactor are in the stored 511 list of active species. If so, then step 515 adds the subject reaction to the active reaction list. If not, then determination junction 514 proceeds to the next reaction in the permissible reactions (full file) input 205 as illustrated at 516 and 512 of loop 513. That is, loop 513 iterates the process (steps 512 to 516) with the next permissible reaction.

When all the permissible reactions from the full list 205 have been processed, the result is an active reaction list 520 (reduced in number from the full list 205). The active reaction list 520 is then fed to the code generator 225 of the MB reactor builder 150 to generate the set of reaction rate equations 152 in equation-oriented format for the reduced reactor model of MB EORXR Block 155.

### Computer Implementation

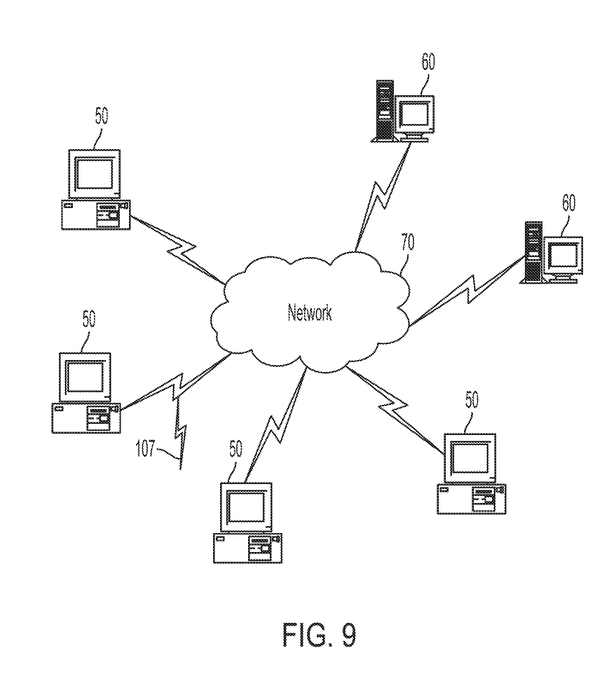

FIG. 9 illustrates a computer network or similar digital processing environment in which the present invention may be implemented.

Client computer(s)/devices 50 and server computer(s) 60 provide processing, storage, and input/output devices executing application programs and the like. Client computer(s)/devices 50 can also be linked through communications network 70 to other computing devices, including other client devices/processes 50 and server computer(s) 60. Communications network 70 can be part of a remote access network, a global network (e.g., the Internet), cloud computing servers or service, a worldwide collection of computers, Local area or Wide area networks, and gateways that currently use respective protocols (TCP/IP, Bluetooth, etc.) to communicate with one another. Other electronic device/computer network architectures are suitable.

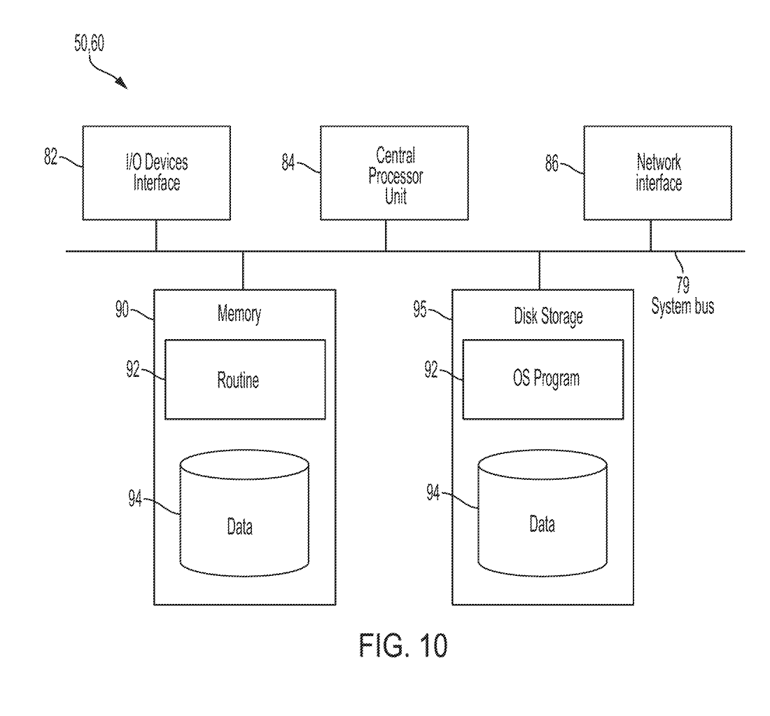

FIG. 10 is a diagram of the internal structure of a computer (e.g., client processor/device 50 or server computers 60) in the computer system of FIG. 9. Each computer 50, 60 contains system bus 79, where a bus is a set of hardware lines used for data transfer among the components of a computer or processing system. Bus 79 is essentially a shared conduit that connects different elements of a computer system (e.g., processor, disk storage, memory, input/output ports, network ports, etc.) that enables the transfer of information between the elements. Attached to system bus 79 is I/O device interface 82 for connecting various input and output devices (e.g., keyboard, mouse, displays, printers, speakers, etc.) to the computer 50, 60. Network interface 86 allows the computer to connect to various other devices attached to a network (e.g., network 70 of FIG. 9). Memory 90 provides volatile storage for computer software instructions 92 and data 94 used to implement an embodiment of the present invention (e.g., input data modules 121, 123, 125; generated pre-estimated thermodynamic properties table 151; MB reactor builder 150 with parser and code generator components 221-225; and reactor model code 152, 155 detailed above). Disk storage 95 provides non-volatile storage for computer software instructions 92 and data 94 used to implement an embodiment of the present invention along with solver and simulator code 170 to 177 as discussed above. Central processor unit 84 is also attached to system bus 79 and provides for the execution of computer instructions.

In one embodiment, the processor routines 92 and data 94 are a computer program product (generally referenced 92), including a computer readable medium (e.g., a removable storage medium such as one or more DVD-ROM's, CD-ROM's, diskettes, tapes, etc.) that provides at least a portion of the software instructions for the invention system. Computer program product 92 can be installed by any suitable software installation procedure, as is well known in the art. In another embodiment, at least a portion of the software instructions may also be downloaded over a cable, communication and/or wireless connection. In other embodiments, the invention programs are a computer program propagated signal product 107 embodied on a propagated signal on a propagation medium (e.g., a radio wave, an infrared wave, a laser wave, a sound wave, or an electrical wave propagated over a global network such as the Internet, or other network(s)). Such carrier medium or signals provide at least a portion of the software instructions for the present invention routines/program 92.

In alternate embodiments, the propagated signal is an analog carrier wave or digital signal carried on the propagated medium. For example, the propagated signal may be a digitized signal propagated over a global network (e.g., the Internet), a telecommunications network, or other network. In one embodiment, the propagated signal is a signal that is transmitted over the propagation medium over a period of time, such as the instructions for a software application sent in packets over a network over a period of milliseconds, seconds, minutes, or longer. In another embodiment, the computer readable medium of computer program product 92 is a propagation medium that the computer system 50 may receive and read, such as by receiving the propagation medium and identifying a propagated signal embodied in the propagation medium, as described above for computer program propagated signal product.

Generally speaking, the term “carrier medium” or transient carrier encompasses the foregoing transient signals, propagated signals, propagated medium, storage medium and the like.

In other embodiments, the program product 92 may be implemented as a so called Software as a Service (SaaS), or other installation or communication supporting end-users.

### EXEMPLIFICATION

### Example 1

The following is a description of a computer-implemented method of modeling chemical reactions in a chemical reactor.

A set of homologous series was used to describe the molecular components in refining hydrocarbon mixtures. An example of molecular components for a hydrocracker (HCR) is shown in FIG. 3. Starting from the feedstock's molecular composition, the molecular composition of the HCR products is derived from HCR reactions and kinetics. Applying hydrocracking chemistries shown in FIG. 4A, we obtain 1366 species and 3186 reactions for this HCR example by Kinetic Modeling Toolkit (KMT) (provided by Klein Research Group (KRG), University of Delaware).

In some embodiments, the molecular level kinetics model can include thousands or tens of thousands of distinct reactions (e.g., 3186 reactions for the hydrocracker example). As a practical matter, it is infeasible to adjust or tune thousands or tens of thousands of kinetic parameters of individual reactions for such a model. To address this, we apply a Linear Free Energy Relationship (LFER) to determine the kinetic parameters in the molecule-based model. The LFER is derived from transition state theory. For each reaction j in a certain reaction family i, the kinetic rate can be expressed as Eq. 6.

ln kᵢⱼ=ln Aᵢ−Eᵢⱼ Eq. 6

Eᵢⱼ=Eᵢ₀+αᵢ·ΔH_{r×n}_{ᵢⱼ}(Polanyi Correlation)

All reactions in one reaction family i have the same pre-exponential factor Aᵢ, and the activation energy Eᵢⱼ is correlated by the Polanyi correlation in terms of an intercept Eᵢ₀, slope αᵢ and the enthalpy change of a reaction ·ΔH_{r×n}_{ᵢⱼ}. The ·ΔH_{r×n}_{ᵢⱼ} is determined by the thermodynamic properties of reactants and products. There are only 3 parameters for each reaction family. Usually, a refining reaction network contains about ten reaction families, so the molecule-based model will only need about 30 parameters to constrain 10,000 reactions. This is practical for users to handle. Typically, the parameters in the LFER are strongly associated with the catalyst but independent of individual species and reactions.

To model heterogeneous catalytic reactions (e.g. HCR, FCC, Reformer), the LHHW rate law was applied in MB EORXR block 155. For refining chemistries, there are many customized empirical terms in the LHHW expression. By summarizing the LHHW rate law format reported from literature and our kinetic expertise, we abstract two generic LHHW rate law expressions to model common refining chemistries. The first generic expression is shown in Eq. 7

$$r = η \cdot k \cdot \frac{DrivingForceGroup}{AdsorptionGroup}$$

r is the rate of certain reaction

$$DrivingForceGroup = {p}_{{H}_{2 }}^{{F}_{fwd}} \cdot {DrivingForce}_{fwd} - {p}_{{H}_{2}}^{{F}_{bwd}} \cdot \frac{{DrivingForce}_{bwd}}{Keq} \\ AdsorptionGroup = {f}_{custom} \cdot ( {p}_{{H}_{2}}^{HPWR} + \sum ( {K}_{i} \cdot {P}_{i} ) ) \\ {K}_{i} = B \cdot {Prop}_{ij } \qquad Eq . 7$$

k is the kinetic rate constant determined by the LFER in Eq. 6.

η is the kinetic multiplier for each reaction (e.g. effectiveness factor)

DrivingForce is determined by stoichiometry for each reaction, by default, F_{fwd}=F_{bwd}

p_{H}_{₂} is the hydrogen partial pressure

Pᵢ is the partial pressure of the component i

Kᵢ is the adsorption constant for compound class i (e.g. aromatic, H₂S, NH₃, etc.). It can be estimated as a correlation with the selected property j of the component i: Propᵢⱼ

B is the coefficient of the Kᵢ correlation.

HPWR is the empirical power item of hydrogen

ƒ_{custom} is a user defined empirical factor.

ƒ_{custom} can be edited by the user with simple math expressions.

The first generic expression is more consistent with the conventional empirical rate law expression in industry. With the detailed molecular composition, we can propose a second generic rate law expression that can elaborate adsorption terms at the molecular level. The second generic expression is developed as shown in Eq. 8. Eq. 8 is an example of the surface control version of LHHW.

$$r = η \cdot {k}_{SR} \cdot \prod {K}_{ireact} \frac{DrivingForceGroup}{AdsorptionGroup}$$

r is the rate of a particular reaction

$$DrivingForceGroup = {p}_{{H}_{2}}^{{F}_{fwd}} \cdot {DrivingForce}_{fwd} - {p}_{{H}_{2 }}^{{F}_{bwd}} \cdot \frac{{DrivingForce}_{bwd}}{Keq} \\ AdsorptionGroup = {( {f}_{custom} \cdot ( {p}_{{H}_{2 }}^{HPWR} + \sum ( {K}_{i} \cdot {P}_{i} ) ) )}^{n} \\ ln {K}_{i} = {B}_{0} + \frac{\sum {B}_{i} \cdot {Prop}_{ij}}{R \cdot Temp} \qquad Eq . 8$$

k_{SR} is the kinetic rate constant of surface control step determined by the LFER in Eq. 6.

η is the kinetic multiplier for each reaction (e.g. effectiveness factor)

DrivingForce is determined by stoichiometry for each reaction, by default, F_{fwd}=F_{bwd}

p_{H}_{₂} is the hydrogen partial pressure

Pᵢ is the partial pressure of the component i Kᵢ is the adsorption constant for an individual molecular component i. It can be estimated as a correlation with the properties of the component i.

K_{ireact} is the adsorption constant for the reactants.

Bᵢ is the coefficient of the Kᵢ correlation. Propᵢⱼ is the selected property of component i.

R is the ideal gas constant

Temp is the given temperature

HPWR is the empirical power item of hydrogen.

n is the power term of the adsorption group.

ƒ_{custom} is a user defined empirical factor. By default: ƒ_{custom}=¹.

ƒ_{custom} can be edited by the user with simple math expressions.

In Eq. 8, the adsorption constant K of each component is expressed as a function of its molecular properties and temperature. A power item n is added to the adsorption group that can flexibly represent the empirical reaction order of the LHHW rate law compared to the first generic expression, the second generic expression provides more detailed description of the adsorption term but requires more variables.

If we set the adsorption group to be unity, both of Eq. 7 and Eq. 8 can be used to model homogeneous reactions without catalyst.

As addressed above, LFER and LHHW rate law terms in MB EORXR are written in a generic format. When we apply the rate law equations in EO, we can write the generic math formulation of the residuals, sparsity patterns, and analytical jacobians in EO format. As a result, a code builder automates coding of rate laws in equation-oriented format (for the example listed below, Aspen EO format was used). As shown in FIGS. 2A-B, the code builder uses the defined set of homologous series of compounds (e.g., FIG. 3), defined set of permissible reactions (e.g., Table 2.1) and the defined properties of reactor compounds (e.g., Table 3) as input data. The code builder parses the properties of the defined series of homologous compounds and creates code to store and call a local thermodynamics property table. The code builder parses the defined set of permissible reactions to obtain the reactants, products, and associated stoichiometric coefficients for the reactants and products. For each reaction, the code builder generates the code for Eq. 6 to Eq. 8. The generated code includes the residuals, sparsity patterns and analytical Jacobians in terms of EO format.

By way of example, FIG. 2A includes a data table and three example reactions. The first column heading is Name, which corresponds to the Species ID of Table 1.1. The entries for LE_C1, LE_C3, and LE_C2 correspond to Light molecule (<C4). The _C1, _C3, and _C2 suffixes indicate one carbon, three carbons, and two carbons, respectively. The remaining column headings of the data table correspond to the Field Names identified in Table 3. For this particular example, the defined set of permissible reactions includes 3,186 reactions. Table 3 lists how many of each type of reaction which can be summed to give the total of 3,186 reactions.

When the code builder parses the properties of the defined series of homologous compounds to create the code to store and call a local thermodynamic table, all properties listed in Table 3 are read from a predefined property file per each compound and stored as a static data table in the memory. The code to estimate the properties in Table 4 for each compound is generated. Following Eq. 3 or Eq. 4, the code to estimate the heat capacity at the given temperature of gas phase or liquid phase for each compound is created first. Then, for each compound, the standard enthalpy of formation in gas phase and the temperature departure function (following classic thermodynamics) in terms of the heat capacity at a given temperature are used to generate the code to estimate the enthalpy of formation in gas phase at the given temperature. Similarly, the standard Gibbs energy of formation in gas phase and the temperature departure function (following classic thermodynamics) in terms of the heat capacity at a given temperature are used to generate the code to estimate the Gibbs energy of formation in gas phase at the given temperature; the standard entropy in gas phase and the temperature departure function (following classic thermodynamics) in terms of the heat capacity at a given temperature are used to generate the code to estimate the entropy in gas phase at the given temperature; the standard enthalpy of formation in liquid phase and the temperature departure function (following classic thermodynamics) in terms of the heat capacity at a given temperature are used to generate the code to estimate the enthalpy of formation in liquid phase at the given temperature; the standard Gibbs energy of formation in liquid phase and the temperature departure function (following classic thermodynamics) in terms of the heat capacity at a given temperature is used to generate the code to estimate the Gibbs energy of formation in liquid phase at the given temperature.

Parsing the defined set of permissible reactions includes obtaining, for each reaction, the reactants, products, and stoichiometric coefficients for those reactants and products. In the first reaction, the reactants are identified by their Species Names: H2 and A6_2N1Ph1HT1_C24. In the first reaction, there is only one product, which is also identified by its Species Names: A6_1N2Ph1SS1_C24. The stoichiometric coefficients are 3.0 for H2, 1.0 for A6_2N1Ph1HT1_C24, and 1.0 for A6_1N2Ph1_SS1_C24. The reaction family (Sat6hTh) informs the rate law. In the Species Name, the suffix “_C24” indicates that the Species has twenty-four carbon atoms. This particular reaction also includes delimiter, which is the “+” symbol. The double-ended arrow indicates that the reaction is reversible.

For each reaction in the defined set of permissible reactions, the process and equation format are the same, but the parameters change.

To simulate a reactor, in addition to the kinetics part, we write equations of mass balance, energy balance and momentum balance. For example, an industrial fixed bed reactor model was implemented in molecule-based equation oriented reactor block. The governing equation is shown in Eq. 9.

$$\frac{{dF}_{A}}{dV} = {ρ}_{CAT} \cdot ( 1 - ɛ ) \cdot {r}_{{Net}_{A }} \qquad Eq . 9$$

ρ_{CAT} is the catalyst particle density in a reactor bed

ε is the void fraction of a reactor bed

F_{A} is the mole flow rate of species A

r_{Net}_{_{A}} is the net generation rate of species A in a reaction bed

To calculate r_{Net} of all species in the model, we apply a matrix operation as shown in Eq. 5

$$[ \begin{matrix}{C}_{11} & {C}_{12} & … & {C}_{1 m} \\ {C}_{21} & … & … & {C}_{2 m} \\ … & {C}_{ij} & … & … \\ {C}_{n 1} & … & … & {C}_{n m}\end{matrix} ] \cdot [ \begin{matrix}{r}_{1} \\ {r}_{2} \\ … \\ \begin{matrix}… \\ …\end{matrix} \\ {r}_{m}\end{matrix} ] = [ \begin{matrix}{r}_{{Net}_{1}} \\ {r}_{{Net}_{2}} \\ … \\ {r}_{{Net}_{n}}\end{matrix} ] \qquad Eq . 10$$

For a complex reaction model containing m reactions and n species,

$$[ \begin{matrix}{C}_{11} & {C}_{12} & … & {C}_{1 m} \\ {C}_{21} & … & … & {C}_{2 m} \\ … & {C}_{ij} & … & … \\ {C}_{n 1} & … & … & {C}_{n m}\end{matrix} ]$$

is a n*m stoichiometric coefficient matrix. A given row i of this matrix indicates the stoichiometric coefficients of species i in reactions 1 to m. Cᵢⱼ is the stoichiometric coefficient of species i in reaction j. If the species i is not involved in reaction j, Cᵢⱼ is zero. If species i is the reactant of reaction j, Cᵢⱼ is equal to the stoichiometric coefficient of species i multiplied by −1. If species i is the product of reaction j, Cᵢⱼ is equal to the stoichiometric coefficient of species i multiplied by +1. The vector [r₁ . . . rₘ] contains the reaction rates of reactions 1 to m that are calculated from Eq. 7 or Eq. 8. The vector [r_{Net}_{₁} . . . r_{Net}_{ₘ}] contains the net generation rates of species 1 to n in a model. Since when the reaction network is given, the stoichiometric coefficient matrix is determined, we use the in-house MB EORXR code builder to automate the coding of Eq. 9 to Eq. 10 in EO format (for the example listed below, Aspen EO format was used). The generated code includes the residuals, sparsity patterns and analytical jacobians for each reaction.

The energy balance equation of a MB EORXR reactor bed is shown in Eq. 11.

$$\frac{dT}{dV} = \frac{1}{F \cdot MixCp} {V}_{p} \sum {r}_{i} \cdot ( - Δ {H}_{{rxn}_{i}} ) + UA ( T - {T}_{c} ) \qquad Eq . 11$$

T is the temperature in the reactor bed

F is the total mole flow rate in the reactor bed.

Vₚ is the volume of the reactor bed.

rᵢ is the reaction rate of reaction i and ΔH_{r×n}_{ᵢⱼ} is the enthalpy change of reaction i

UA is the heat transfer coefficient to the environment and T_{c} is the environment temperature.

For an adiabatic reactor (e.g. HCR), the second term of Eq. 11 can be ignored.

The momentum balance of MB EORXR in a reactor bed is simplified to consider one dimension effect and Ergun equation shown in Eq. 12 is applied:

$$\frac{dP}{dZ} = - f \cdot \frac{ρ \cdot {u}_{s}^{2}}{{d}_{p}} \\ f = \frac{1 - ɛ}{{ɛ}^{3}} \cdot ( 1.75 a + 150 b \frac{( 1 - ɛ )}{Re} ) \qquad Eq . 12$$

P is the pressure in the reactor bed

ρ is the density of the stream

uₛ is the superficial velocity

dₚ is the diameter of the catalyst particle in the reactor bed

ε is the void fraction of a reactor bed

f is the friction factor

Re is Reynold number

a and b are turbulent and laminar correction coefficients. By default, a=b=1

Essentially, in this example, a reactor bed simulation solves a set of ordinary differential equations sets illustrated from Eq. 6 to Eq. 12. To simulate the above ordinary differential equations sets in equation-oriented solver, the method of Orthogonal Collocation on Finite Elements (OCFE) is used in molecule based equation oriented reactor block and a 4^{th} order Runge Kutta method is applied to get the initial solutions for OCFE in order to improve the convergence performance of EO solver.

The properties used in MB EORXR are a set of selected thermodynamic properties and physical properties shown in Table 3. The comprehensive thermodynamics model is not required in MB EORXR. The properties of the molecular compositions in MB EORXR are constant values, so a local property table is created to load in MB EORXR. The data of the local property table are provided by MC. Therefore, MB EORXR computational burden of property estimation is reduced significantly and thus the number of species and reactions MB EORXR can handle overcomes the limitation of conventional EO models.

All of the jacobians of the equations from Eq. 6 to Eq. 12 are written in analytical format in order to get the best performance when MB EORXR is used to solve the large-scale problem (O (10000) species, O (50000) reactions).

Because the rigorous thermodynamic calculations are not required, MB EORXR can directly communicate with a flowsheet simulator without additional overhead. Relevant transition between the detailed molecular composition of the reactor and assay components of the simulator is straightforward. As a result, the performance of solving the model is increased. The new framework of Molecule-Based Reactor is shown in FIG. 1.

For various refining chemistries (e.g., HCR, Reformer, FCC), the models have different Eq. 6 to Eq. 10. As illustrated in FIG. 2, in addition to automating the code of Eq. 6 to Eq. 8, the MB EORXR builder can automate the coding of Eq. 9 to Eq. 10 in EO format (residual, sparsity pattern, and analytical Jacobian after parsing the reactions and compounds through the input file of the reaction list illustrated in the previous section. MB EORXR can create reactors of various refining chemistries without tedious hard coding. The following is an example of the HCR MB model.

Starting from a molecular representation of feedstock shown in FIG. 3, a representative molecule-based hydrocracker network is obtained via KMT. The statistics of the HCR reaction network are shown as Table 2.1. 1366 species and 3186 reactions are obtained for this model.

The local property table of 1366 species are calculated or determined. Using the 3186 reactions and the local property table of 1366 species, the in-house MB EORXR code builder generates all necessary code for the reaction network represented by Eq. 6 to Eq. 10 and integrates with the code specific to the reactor represented by Eq. 11 to Eq. 12 to compile a MB EORXR HCR reactor block. The performance of a single bed HCR reactor is shown in Table 5. The example listed below used Aspen EO as the solver for MB EORXR.

TABLE 5
Model performance of a single bed molecule-based hydrocracker
full model.

| | Full MB HCR |
|---|---|
| | Model |
| Number of variables | 111862 |
| Number of equations | 107208 |
| The number of non-zero | 874196 |
| variables | |
| Typical solving time (sec) | 8 |
| Average time of one iteration | 2.7 |
| (sec) | |

Table 5 shows the model size of this single bed MB HCR model. This model describes a detailed HCR with 1366 species and 3186 reactions, and the complexity of this model is larger than a six bed HCR using our conventional HCR model. However, a single bed conventional HCR only has 97 species and 177 reactions. The model resolution of MB HCR is over 13 times the conventional HCR, but the solving time of a single bed MB HCR is quite acceptable. Using Runge Kutta method for SM initialization, the MB HCR model can accelerate the convergence steps and take ˜2.5 secs for each iteration. For both R&D and plant users, this performance is very practical for a single bed reactor model.

However, when a user sets up a complex flowsheet with multiple reactor beds in EO, the size of the total model becomes much larger and the solution time is slowed down too much. A performance test of a typical 4 bed hydrocracker (HCR) MB reactor model is shown in Table 6.

TABLE 6
Model performance of a four-bed molecule-based hydrocracker full
model in a standard desktop (CPU 3.4 GHZ).

| | Full MB HCR |
|---|---|
| | Model |
| The number of variables | 482406 |
| The number of equations | 459698 |
| The number of non-zero | 3643172 |
| variables | |
| Typical solving time (sec) | 111.5 |
| Average time of one | 18.6 |
| iteration (sec) | |

Table 6 shows that the size of a four-bed HCR model is extremely large. The model has ˜0.45 million (M) variables and equations and ˜3.6 M non-zero variables. Due to the limitation of 32 bit applications (2 GB to 4 GB of memory available depending on application and OS), the model is almost at the model size limit for 32 bit applications. Moreover, the solution time is affected by the model size. Although the model has good calculation time, the solver computational time becomes significantly longer when the size of model is increased to a very large scale. The average time of one iteration costs ˜18.6 secs and the total solution time of the four-bed MB HCR model is on the order of minutes instead of on the order of seconds. Even though the memory limitation can been solved by upgrading to a 64 bit application, industrial users may require a more rapid solution, such as for Real Time Optimization (RTO).

### Reduced Model

To further improve the solution time, we develop a model reduction approach, which is particularly useful for improving performance for large scale multiple bed reactor models.

To accomplish MB EORXR model reduction, we considered two steps: the function that allows users to run MB models with controllable model size and the strategy of the model reduction without losing important kinetic activities.

FIG. 5 is a flowsheet describing control of the size of a molecule-based reactor model.

The number of reactions is derived from the number of species with a set of chemistry rules. We can start with the selection of species to reduce both number of species and reactions. Based on the strategy of model reduction, a list of species involved with the reduced model is created, which are referred to as the active species. Each reaction in a full reaction network is analyzed. For a given reaction i, all species involved in reaction i are parsed and checked. If all species in reaction i are active species, reaction i is added to the active reaction list; otherwise this reaction is skipped. Then the program will continue to check reaction i+1. After traversing all reactions in the model, the active reaction list is obtained. The above procedure is the pre-processing of MB EORXR. After this pre-processing, the active reaction list and active species list are sent to the main part of MB EORXR. The MB EORXR will use the reactions in the active reaction list and species in the active species list to create the necessary variables and equations of a reactor block.

Since the species and reactions involved in this model reduction are determined from a given user model reduction strategy, this model reduction can be loaded dynamically without hard-coding and re-compiling of the MB EORXR block. Therefore, users can easily test different model reduction strategies by simply using one MB EORXR block to obtain the best reduced model. In addition, if the active species and reactions are the full list, this model reduction switches back to the MB full model.

To get the benefits of molecular level modeling, we want to keep the key reactivity of the MB model when we use the MB reduced model. This provides guidance on how to choose a model reduction strategy. An alternative approach is to analyze the essential nature of chemistries and kinetics of the model and conduct a strategic model reduction solution. The leading examples are EXXONMOBIL SOL and Attribute Reaction Model (ARM) from Klein Research Group (KRG). The latter approach requires less complexity of numerical analysis but is more dependent on the user expertise and experience of chemistry and chemical engineering. For an industrial solution, the latter approach is selected as the model reduction strategy for creating the MB EORXR block. A strategic carbon-number-based model reduction is developed subsequently.

A complex hydrocarbon mixture usually contains a large carbon number range (e.g. 1˜40), the juxtapositions of molecular types and the large carbon number extension (e.g. 40) is a combinatorial problem and leads to a large number of molecules. The number of molecules is significantly affected by the carbon number extension in each series. If we reduce the carbon number extension in each series, the number of species in a reactor model can be decreased significantly.

Besides the analysis of molecular composition, we also analyze the reaction network of the MB model.

From Table 2.1, we can observe that the cracking-related reactions (DEALK and PCR) constitute more than 50% of reactions in this example. If we can reduce the carbon number extensions in each series, the number of reactions in a reactor model can be decreased significantly.

In addition, as we describe above, the molecular type is the key factor for each series. If we keep all molecular types intact and only reduce the carbon number extension, we will not lose important reactivity and thermodynamic information and thus the prediction of product yields and product quality properties can be maintained as with the full model.

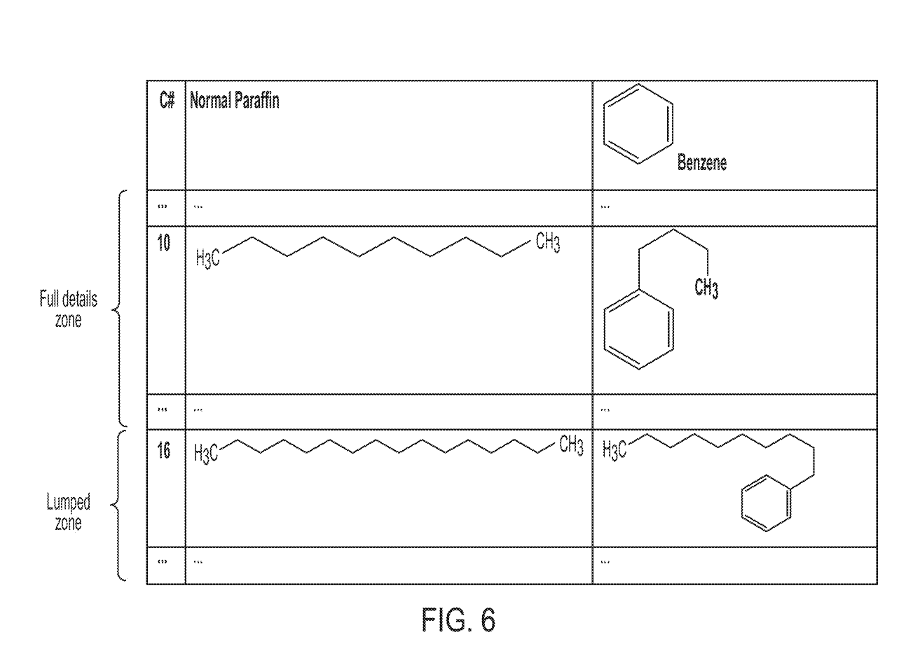

When the details of carbon number extension of molecular compositions are reduced, we will lose some information. It is very important to expose the criteria to control the details of the carbon number extension we want to keep. The first criterion is the minimum lump carbon number Cₗᵤₘₚₘᵢₙ. As shown in FIG. 6, a horizontal line can be set up across all molecular series. Above this line, the molecular composition is in the fully detailed zone. The species of each series whose carbon number is smaller than Cₗᵤₘₚₘᵢₙ is kept in the model. Below this line, the molecular composition is in the lumped zone; species of each series whose carbon number is larger than Cₗᵤₘₚₘᵢₙ are reduced to a set of limited carbon lumps. This criterion can let users determine to keep a full-detailed zone of molecular representation in the reduced model in order to maintain maximum details of the very important range in a hydrocarbon mixture. In the example of the HCR model, we chose Cₗᵤₘₚₘᵢₙ=18, which means all species up to C18 are kept intact in the reduced model. C1˜C18 is the range of fractions including naphtha, kerosene and diesel, which are the high valued products of refining and are of highest interest to refining users.

The second criterion to control the details of the model is the carbon number interval of the species: Cₗᵤₘₚᵢₙₜₑᵣᵥₐₗ in the lumped zone. The species of each molecular series in the lumped zone are reduced to a set of carbon lumps. The lumped carbon numbers are selected from the continuous carbon number range discretized by Cₗᵤₘₚᵢₙₜₑᵣᵥₐₗ. For example, if Cₗᵤₘₚᵢₙₜₑᵣᵥₐₗ is set as 4, a carbon number range of 18˜30 is discretized to 18,22,26,30. The number of species in this range is reduced from 13 to 4. How the Cₗᵤₘₚᵢₙₜₑᵣᵥₐₗ is selected determines the size and the accuracy of the MB reduced model. The optimal selection of Cₗᵤₘₚᵢₙₜₑᵣᵥₐₗ is a key to this reduction strategy.

For the example of the HCR MB model, we selected 4 as Cₗᵤₘₚᵢₙₜₑᵣᵥₐₗ via analyzing reactions and species of the HCR based on our kinetic expertise and experience. As shown in FIG. 3, the reactions of HCR include acid chemistries and metal chemistries.

The reactions that solely occur on sub-structures of molecular types, that is the carbon number remains the same in reactants and products, are not affected by the carbon number based model reduction (e.g. Saturation 6H, Saturation 4H, HDS, and HDN). The major reactions affected by this reduction are cracking relevant reactions such as Dealkylation/Sidechain cracking and paraffin hydrocracking. Those reactions belong to the acid chemistry mechanism. Due to the relative thermochemical stabilities of different carbenium ions, the minimum cracked products are C4/C3 paraffins. The reactions that change both the molecular types and carbon number in reactants and products also contribute to the carbon number based reduction such as RingOpen. For the RingOpen reaction, a C4 paraffinic segment is created from an aggregated ring structure. The minimum product segment having the highest probability in HCR is C4. As a result, we can obtain the best derivative of carbon number continuity in the lumped species if we choose 4 as Cₗᵤₘₚᵢₙₜₑᵣᵥₐₗ.

Although we do not lose the key reactivity of the MB model by keeping the molecular type intact, we still need to figure out a way to track the carbon number extension back from the lumped post-reaction species. In general refining chemistries, we find from the literature that the carbon number distribution of molecular compositions in feedstocks and products are both continuous. The carbon number profile of hydrocarbon feeds and products is also continuous in its first and second derivatives. Therefore, by giving sufficient carbon number lumped species for each molecular series, we can apply a numerical cubic spline function to interpolate/extrapolate the full carbon number details of each series. As a result, the full molecular details of products in MB reduced models are reversibly mapped back from the lumped species of the reactor effluent. An example of the MB HCR reduced model is shown subsequently.

We applied the procedure 150A of FIG. 5 to a full HCR MB model including 1366 species and 3186 reactions with the above criteria of carbon number reduction. As a result, a reduced model with 567 active species and 973 active reactions is obtained. The size of this MB reduced model is around one third of the MB full model. Table 7 shows the model performance of the single bed MB HCR models.

TABLE 7
Model performance of single bed molecule-based hydrocracker models in
a standard desktop (CPU 3.4 GHZ).

| | Full MB HCR | Reduced MB HCR |
|---|---|---|
| | Model | Model |
| The number of variables | 111862 | 45142 |
| The number of equations | 107208 | 40370 |
| The number of non-zero | 874196 | 310863 |
| variables | | |
| Typical solving time (sec) | 8 | 1.8 |
| Average time of one | 2.7 | 0.6 |
| iteration (sec) | | |

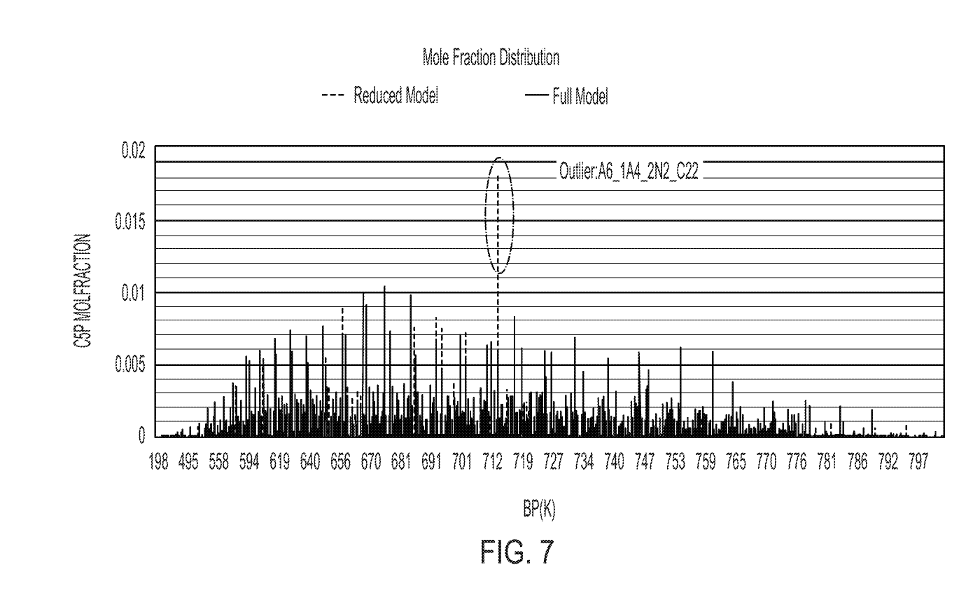

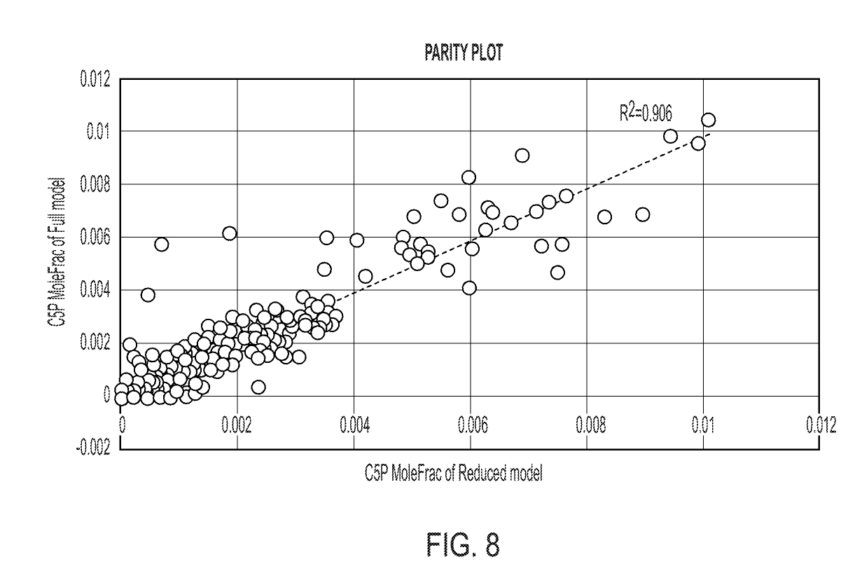

Shown in Table 7, the solving speed of a single bed MB reduced HCR model is around 5 times faster than a single bed MB full HCR model. Another aspect we consider about the reduced model is the accuracy of the model estimations. Given the same kinetic parameters, feed composition and reaction conditions, the MB full HCR model and the MB reduced HCR model results are shown in Table 8, FIG. 7 and FIG. 8.

Table 8 shows the key HCR model results of the MB full HCR and the MB reduced model. The temperature rise of a reactor bed and the removal wt % of aromatics are close and the removal wt % of sulfur contents is also acceptable. The results of the HCR model remain consistent between the full model and the reduced model.

TABLE 8
Comparisons of key model specs between MB full HCR and
MB reduced HCR.

| | Full Model | Reduced Model |
|---|---|---|
| Reactor Temperature Rise (° C.) | 19.22 | 20.26 |
| Aromatic wt % change | 7.139 | 6.975 |
| Sulfur wt % change | 0.7503 | 0.6808 |

FIG. 7 shows the distributions of mole fractions versus boiling points of the MB full HCR and the MB reduced HCR model. There is good agreement between the full model and the reduced model except one outlier component. This outlier is an intermediate species from saturation reactions and dealkylation of polynuclear aromatics. The cracking reactions in the full carbon range model are still not fully comprehensive for hydrocracking but a reduced optimal set of cracking reaction paths was created by KMT. This may cause the information of carbon number extension to be missed by the model reduction. This issue may be addressed by re-optimizing the full model reaction network by KMT and improving our model reduction strategy.

FIG. 8 is a parity plot of the mole fractions profile between the MB full HCR and the MB reduced HCR. The molecular fraction profile shows good consistency between the full model and the reduced model. The results of the MB reduced model are not perfect but are a good approximation for industrial applications. In this example, we did not change the kinetic parameters of the MB reduced HCR model. It shows that the parameters are not very dependent on the carbon number based model reduction strategy. But we also can improve the results of the MB reduced HCR model by fine-tuning the parameters.

The purpose of the MB reduced model is to increase the performance (e.g., reduce solving time) of multiple bed MB reactor models. As a comparison, the performance of four bed HCR reactor models is shown in Table 9.

TABLE 9
Equation-Oriented solving performance of four bed hydrocracker
reactor models.

| | Full | | |
|---|---|---|---|
| | MB HCR | Reduced MB | Conventional |
| | Model | HCR Model | HCR Model |
| Number of variables | 482406 | 215526 | 59860 |
| Number of equations | 459698 | 192346 | 56935 |
| Number of non-zero variables | 3643172 | 1389840 | 688680 |
| Typical solving time (sec) | 111.5 | 25.4 | 15.7 |
| Average time of one iteration | 18.6 | 3.7 | 1.96 |
| (sec) | | | |

From Table 9, the first column shows the model performance of a four bed MB HCR full model; the second column shows the model performance of a four bed MB HCR reduced model; and the third column show the model performance of a four-bed conventional HCR model (97 species and 177 reactions). The size of a four-bed MB reduced model is much smaller than that of a full model. The DMO computational time of a four bed MB reduced model is significantly faster than that of a full model. The average time of one iteration costs ˜3.7 secs and the total solution time of four bed MB HCR model is O (secs). Compared with the conventional model, the four bed MB reduced HCR model has the same order magnitude of solving performance.

In summary, by using MB EORXR, users can simulate a single bed MB reactor model of complex refining chemistries on a practical time scale. The detailed molecular information of the MB EORXR model allows the user to capture the intrinsic kinetic parameters of complex chemistries, reveal the essential nature of reaction mechanisms and get better predictions of product yields and properties.

The MB reduced model function in MB EORXR provides the user a flexible option to control the model size from full detail to a limited number of species. As a result, users can apply different model reduction strategies to fulfill different purposes (e.g. RTO, simplified model for planning etc.). The carbon number based model reduction strategy can effectively reduce the size of the full MB model, and increase the model solving performance without losing important MB reactivity information. The numerical spline function is able to reversibly map the full molecular composition to the products from the effluents of the MB reduced reactor model. The comparison results of the MB full model and the MB reduced model of HCR show good agreement between them and the MB reduced model is a good approximation for industrial applications. The MB reduced HCR model maintains full molecular details while having the same computational performance as the conventional HCR model. MB reduced model is a practical solution to apply molecular kinetics and reaction models to complex industrial applications that require fast solution.

### Example 2

The following is a description of a computer-implemented method of modeling chemical reactions in a chemical reactor. This example was created by MB EORXR for Hydrocracking/hydrotreating.

In order to handle the heavy oil (sometimes referred to as petroleum residue or petroleum resid), the hydrocracking/hydrotreating model of Example 1 was extended. Additional species (molecular types) that represent the molecular structures of resid were included. An additional reaction family was included to break up the archipelago structures of resid to the smaller molecules: inter-core linkage cracking (ILCR). In addition, sulfide species were included to obtain a more comprehensive representation of hydrocracking components. A distinct reaction family to remove the sulfur atom in sulfides (HS) was also added to the model. In addition, the number of dealkylation and isomerization reactions was increased in order to obtain a more granular product distribution. As a result, an extended hydrocracking/hydrotreating example model handling the feedstock ranging from naphtha to resid was created referred as “New MB HCR model.” The statistics of a single bed Full New MB HCR model are shown in Table 10.

TABLE 10
Model performance of a single bed new MB HCR full model
in a standard desktop (CPU 3.4 GHZ).

| | Full New MB HCR model |
|---|---|
| Number of components | 2431 |
| Number of reactions | 5734 |
| Number of variables | 284035 |
| Number of equations | 275731 |
| The number of non-zero | 2055331 |
| variables | |
| Typical solving time (sec) | 24.4 |
| Average time of one iteration | 3.2 |
| (sec) | |

The resolution of the Full New MB HCR is greater than the MB HCR model in Example 1, and the Full New MB HCR includes a wider range of molecules that can be present in a petroleum feedstock for hydrocracking/hydrotreating. In particular, more molecules that are typically found in the resid are included. Users can choose a sub range of the components and reactions from the Full New MB HCR model to increase solving performance if they only need to model a certain oil fraction (e.g. diesel, gasoil, etc.). For example, to appropriately balance speed vs. granularity of the data, a user may select an appropriate lower and upper bound for the carbon number. In order to achieve greater speeds, the user can select only those compounds having between one and forty carbon atoms (a lower bound of one carbon atom and an upper bound of forty carbon atoms). For improved resolution, a user can select compounds having between one and eighty carbon atoms (a lower bound of one carbon atom and an upper bound of eighty carbon atoms). Users can select an appropriate subset of the carbon number range, depending on their particular requirements and demands.

In this example, we utilized a 64 bit EO solving engine, which removed the limitations of number of variables and equations in the Example 1, which was implemented using a 32 bit EO solver. As a result, the 64 bit EO solver has increased memory, thereby permitting an increased number of reactions in the set of permissible reactions. As a result, the 64 bit EO solver can accommodate, for example, larger scale models and/or more reactor beds. To create a performance benchmark, we set up a hydrocracking flowsheet with 12 reactor beds created by the Full New MB HCR model to test the performance of the new MB HCR model in a large scale hydrocracking flowsheet. Both full and reduced models were tested and the results are shown in Table 11.

TABLE 11
Equation-Oriented solving performance of 12 bed hydrocracker reactor
using new MB HCR models in a standard desktop (CPU 3.4 GHZ).

| | Full New MB | Reduced New MB |
|---|---|---|
| | HCR Model | HCR Model |
| Number of variables | 2886584 | 1148252 |
| Number of equations | 2786906 | 1046894 |
| Number of non-zero variables | 21286342 | 7528282 |
| Typical solving time for | 427.5 | 84.5 |
| simulation (sec) | | |
| Average time of one iteration | 35.6 | 7 |
| for simulation (sec) | | |
| Typical solving time for | 529.2 | 152.1 |
| calibration (sec) | | |
| Average time of one iteration | 37.8 | 9.5 |
| for calibration (sec) | | |

In addition to testing the performance of the simulation, a calibration test was performed in this example. Calibration involves tuning the kinetic parameters of the reactor model to match plant measurements. In the calibration, a least squares objective function is created in terms of the measurements of the products. The EO solver adjusts the kinetic parameters to minimize the objective function in order to match the product information closely and obtain the optimal kinetic parameters. Although the calibration was an optimization problem, the solution time of the calibration is the same order of magnitude as the simulation. The full model can be solved on the order of approximately 500 seconds, which is acceptable for selected advanced users. The reduced model can be solved more quickly (approximately 1-2 minutes), which is sufficient for an application needing a more rapid solution such as Real Time Optimization (RTO).

### Example 3

The following is a description of a computer-implemented method of modeling chemical reactions in a chemical reactor. This example was created by MB EORXR for Hydrocracking/hydrotreating.

In order to handle the chemistry of Polynuclear Aromatic Hydrocarbon (PAH) molecules in the heavy oil (sometimes referred to as petroleum residue or petroleum resid), the hydrocracking/hydrotreating model of Example 2 was extended. An additional reaction family, aromatic ring condensation (ARConden), was included to formulate a PAH molecule with higher ring number by condensing two smaller PAH molecules. The molecular types of Table 1.2 were included as molecular species that derive from aromatic ring condensation of resid. As a result, an extended hydrocracking/hydrotreating example model handling the feedstock ranging from naphtha to resid was created referred as “Full New MB HCR model 2.” The statistics of a single bed Full New MB HCR model 2 are shown in Table 12.

TABLE 12
Model performance of a single bed full new MB HCR model 2 in a
standard desktop (CPU 3.4 GHZ).

| | Full New MB HCR model 2 |
|---|---|
| | (single bed) |
| Number of components | 2696 |
| Number of reactions | 5825 |
| Number of variables | 293258 |
| Number of equations | 291209 |
| The number of non-zero | 2403484 |
| variables | |
| Typical solving time (sec) | 56.3 |
| Average time of one iteration | 4.3 |
| (sec) | |

The resolution of the Full New MB HCR model 2 is greater than the MB HCR model in Example 1, and the Full New MB HCR includes a wider range of molecules that can be present in a petroleum feedstock for hydrocracking/hydrotreating. In particular, more molecules that are typically found in the resid are included. Users can choose a sub range of the components and reactions from the Full New MB HCR model 2 to improve solving performance if they only need to model a certain oil fraction (e.g. diesel, gasoil, etc.). For example, to appropriately balance speed vs. granularity of the data, a user may select an appropriate lower and upper bound for the carbon number. In order to achieve greater speeds, the user can select only those compounds having between one and forty carbon atoms (a lower bound of one carbon atom and an upper bound of forty carbon atoms). For improved resolution, a user can select compounds having between one and eighty carbon atoms (a lower bound of one carbon atom and an upper bound of eighty carbon atoms). Users can select an appropriate subset of the carbon number range, depending on their particular requirements and demands.

In this example, a 64 bit EO solving engine was utilized, which removed the limitations of number of variables and equations in Example 1, which was implemented using a 32 bit EO solver. As a result, the 64 bit EO solver has increased memory, thereby permitting an increased number of reactions in the set of permissible reactions. As a result, the 64 bit EO solver can accommodate, for example, larger scale models and/or more reactor beds. To create a performance benchmark, we set up a hydrocracking flowsheet with four reactor beds created by the Full New MB HCR model 2 to test the performance of the Full New MB HCR model 2 in a large scale hydrocracking flowsheet. Both full and reduced models were tested and the results are shown in Table 13.

TABLE 13
Equation-Oriented solving performance of four bed hydrocracker
reactor using new MB HCR model 2 in a standard desktop
(CPU 3.4 GHZ).

| | Full New MB | Reduced New MB |
|---|---|---|
| | HCR Model 2 | HCR Model 2 (four |
| | (four beds) | beds) |
| Number of variables | 1186684 | 556584 |
| Number of equations | 1165669 | 523301 |
| Number of non-zero variables | 9104806 | 3726534 |
| Typical solving time for | 363 | 74.46 |
| simulation (sec) | | |
| Average time of one iteration | 181.5 | 14.7 |
| for simulation (sec) | | |

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1. A computer-implemented method of modeling chemical reactions in a chemical reactor, the method comprising:
i) representing reactor compounds by defining a set of homologous series of compounds in the reactor, each homologous series within the set comprising a molecular type and a carbon number, the carbon number having an upper bound and a lower bound range;
ii) defining a set of permissible reactions for the defined set of homologous series of compounds;
iii) defining properties of the reactor compounds;
iv) generating and locally-storing pre-estimated thermodynamic properties based on the defined set of homologous series of compounds;
v) automatically coding a set of reaction rate equations in equation-oriented format based on the defined set of homologous series of compounds, the defined set of permissible reactions, the defined properties of the reactor compounds, and the generated and locally-stored pre-estimated thermodynamic properties,
the automatically coded set of reaction rate equations forming a model of chemical reactions in the chemical reactor.

2. The method of claim 1, wherein the molecular type of the homologous series includes any combination of one or more of: molecular hydrogen (H₂); normal paraffin; pyrrole; benzene; pyridine; cyclohexane; thiophene; tetralin; benzothiophene; indole; naphthalene; quinolone; decalin; tetradecahydrophenanthrene; fluorene; tetrahydrophenanthrene; phenanthrene; benzoquinoline; octadecahydrochrysene; chrysene; naphthoquinoline; picene; naphthobenzothiophene (hard sulfur removal); naphthobenzothiophene (not as hard sulfur removal); dibenzothiophene (hard sulfur removal); dibenzothiophene (not as hard sulfur removal); carbazole; benzocarbazole; light molecule (<C4); tetrahydropyrrole; tetrahydrothiophene; dihydrobenzothiophene; dihydroindole; tetrahydroquinoline; octahydrophenanthrene; tetrahydrobenzoquinoline; tetrahydrochrysene; tetrahydronaphthoquinoline; tetrahydropicene; phenylnaphthalene; hydrogen sulfide; hexahydronaphthobenzothiophene; tetrahydronaphthobenzothiophene (hard sulfur removal); tetrahydronaphthobenzothiophene (not as hard sulfur removal); biphenyl; hexahydrodibenzothiophene; hexahydrocarbazole; hexahydrobenzocarbazole; tetrahydrobenzocarbazole; octahydrobenzothiophene; ammonia; octahydroindole; octahydrobenzoquinoline; octahydropicene; cyclohexylnaphthalene; decahydronaphthobenzothiophene; cyclohexylbenzene; dodecahydrodibenzothiophene; decahydronaphthobenzoquinoline; iso paraffin with one branch; tetradecahydrobenzoquinoline; hexahydrofluorene; iso paraffin with multiple branches; sulfide/mercaptan; tetrahydrobenzoquinoline; tetrahydronaphthoquinoline; octahydronaphthoquinoline; dodecahydrocarbazole; hexadecahydronaphthobenzothiophene; bicyclohexyl; hexadecahydronaphthoquinoline; cyclohexyldecalin; dodecahydronaphthoquinoline; naphthalene connected with chrysene; naphthalene connected with dibenzothiophene; phenanthrene connected with chrysene; phenanthrene connected with naphthobenzothiophene; phenanthrene connected with dibenzothiophene; chyrsene connected with chrysene; chyrsene connected with naphthobenzothiophene; chyrsene connected with picene; picene connected with picene; naphthobenzothiophene connected with picene; naphthobenzothiophene connected with naphthobenzothiophene; and dibenzothiophene connected with naphthobenzothiophene.

3. The method of claim 1, wherein the molecular type of the homologous series includes any combination of one or more of: normal paraffin; iso paraffin with one branch; and iso paraffin with multiple branches.

4. The method of claim 1, wherein the molecular type of the homologous series includes any combination of one or more of: naphthalene fused with naphthalene; naphthalene fused with benzothiophene; naphthalene fused with indole; naphthalene fused with quinoline; quinoline fused with quinoline; benzothiophene fused with quinoline; indole fused with quinoline; biphenyl fused with naphthalene; biphenyl fused with quinoline; benzothiophene fused with benzothiophene; benzothiophene fused with indole; biphenyl fused with benzothiophene; indole fused with indole; biphenyl fused with indole; phenanthrene fused with naphthalene; phenanthrene fused with quinoline; phenanthrene fused with benzothiophene; phenanthrene fused with indole; phenanthrene fused with phenanthrene; biphenyl fused with phenanthrene; benzoquinoline fused with quinoline; benzoquinoline fused with benzothiophene; benzoquinoline fused with indole; benzoquinoline fused with phenanthrene; benzoquinoline fused with benzoquinoline; biphenyl fused with benzoquinoline; dibenzothiophene (hard sulfur removal) fused with naphthalene; dibenzothiophene (hard sulfur removal) fused with quinoline; dibenzothiophene (hard sulfur removal) fused with benzothiophene; dibenzothiophene (hard sulfur removal) fused with indole; dibenzothiophene (hard sulfur removal) fused with phenanthrene; dibenzothiophene (hard sulfur removal) fused with benzoquinoline; dibenzothiophene (not hard sulfur removal) fused with naphthalene; dibenzothiophene (not hard sulfur removal) fused with benzoquinoline; dibenzothiophene (not hard sulfur removal) fused with benzothiophene; dibenzothiophene (not hard sulfur removal) fused with indole; dibenzothiophene (not hard sulfur removal) fused with phenanthrene; dibenzothiophene (not hard sulfur removal) fused with benzoquinoline; carbazole fused with naphthalene; carbazole fused with quinoline; carbazole fused with indole; carbazole fused with phenanthrene; carbazole fused with benzoquinoline; biphenyl fused with chrysene; biphenyl fused with naphthoquinoline; phenylnaphthalene fused with phenylnaphthalene; biphenyl fused with phenylnaphthalene; naphthobenzothiophene (hard sulfur removal) fused with phenylnaphthalene; chrysene fused with naphthalene; chrysene fused with quinoline; chrysene fused with benzothiophene; chrysene fused with indole; chrysene fused with phenanthrene; chrysene fused with benzoquinoline; chrysene fused with phenylnaphthalene; chrysene fused with benzothiophene1; chrysene fused with indole1; naphthoquinoline fused with quinoline; naphthoquinoline fused with benzothiophene; naphthoquinoline fused with indole; naphthoquinoline fused with benzoquinoline; naphthoquinoline fused with phenylnaphthalene; naphthoquinoline fused with benzothiophene1; naphthoquinoline fused with indole1; naphthobenzothiophene (hard sulfur removal) fused with benzothiophene; naphthobenzothiophene (hard sulfur removal) fused with indole; naphthobenzothiophene (hard sulfur removal) fused with naphthalene; naphthobenzothiophene (hard sulfur removal) fused with benzoquinoline; naphthobenzothiophene (hard sulfur removal) fused with biphenyl; naphthobenzothiophene (not hard sulfur removal) fused with benzothiophene; naphthobenzothiophene (not hard sulfur removal) fused with indole; naphthobenzothiophene (not hard sulfur removal) fused with naphthalene; naphthobenzothiophene (not hard sulfur removal) fused with benzoquinoline; naphthobenzothiophene (not hard sulfur removal) fused with phenylnaphthalene; benzocarbazole (not hard sulfur removal) fused with indole; benzocarbazole (not hard sulfur removal) fused with naphthalene; benzocarbazole (not hard sulfur removal) fused with quinoline; benzocarbazole (not hard sulfur removal) fused with phenylnaphthalene; picene fused with naphthalene; picene fused with quinoline; picene fused with biphenyl; naphthobenzothiophene (not hard sulfur removal) fused with biphenyl; picene fused with benzothiophene; picene fused with indole; picene fused with phenanthrene; picene fused with benzoquinoline; picene fused with phenylnaphthalene; picene fused with benzothiophene1; picene fused with indole1; picene fused with phenanthrene1; picene fused with benzoquinoline1; and picene fused with phenylnaphthalenel.

5. The method of claim 1, wherein the automatically coded set of reaction rate equations includes Langmuir-Hinshelwood-Hougen-Watson (LHHW) rate laws.

6. The method of claim 1, wherein generating and locally storing the pre-estimated thermodynamic properties comprises solving equations of kinetic rate and constraining kinetic rate constants by a linear free energy relationship (LFER).

7. The method of claim 1, wherein the automatically coded set of reaction rate equations comprises one or more of residuals, sparsity patterns, and analytical Jacobians in equation-oriented format.

8. The method of claim 1, wherein the defined properties of the reactor compounds include any combination of one or more of: molecular weight; total number of carbon atoms; total number of hydrogen atoms; total number of side chains; total number of aromatic rings; total number of naphthenic rings; total number of thiophenic rings; total number of pyrrolic rings; total number of pyridenic rings; total number of sulfur atoms; total number of nitrogen atoms; total number of oxygen atoms; total number of aromatic carbon atoms; total number of naphthenic carbon atoms; total number of paraffinic carbon atoms; total number of naphthenic six-carbon rings; total number of naphthenic five-carbon rings; boiling point; density; standard enthalpy of formation in gas phase; standard Gibbs free energy of formation in gas phase; a gas phase heat capacity coefficient; heat of vaporization; standard enthalpy of formation in liquid phase; a liquid phase heat capacity coefficient; a viscosity coefficient; and molecular type.

9. The method of claim 1, wherein the defined properties of reactor compounds include any combination of one or more of: total number of carbon atoms, total number of hydrogen atoms, total number of aromatic rings, total number of naphthenic rings, total number of thiophenic rings, total number of pyrrolic rings, total number of pyridenic rings, total number of sulfur atoms, total number of nitrogen atoms, total number of oxygen atoms, standard Gibbs free energy of formation, and standard enthalpy of formation.

10. The method of claim 1, wherein the defined properties of reactor compounds include any combination of one or more of total number of carbon atoms, total number of hydrogen atoms, standard Gibbs free energy of formation, and standard enthalpy of formation.

11. The method of claim 1, wherein the generated and locally-stored pre-estimated thermodynamic properties include any combination of one or more of: enthalpy of formation in gas phase at given temperature; Gibbs free energy of formation in gas phase at given temperature; gas phase heat capacity at given temperature; entropy in gas phase at given temperature; heat of vaporization; enthalpy of formation in liquid phase at given temperature; and liquid phase heat capacity at given temperature.

12. The method of claim 1, wherein the generated and locally-stored pre-estimated thermodynamic properties include any combination of one or more of: enthalpy of formation in gas phase at given temperature; Gibbs free energy of formation in gas phase at given temperature; gas phase heat capacity at given temperature; and entropy in gas phase at given temperature.

13. The method of claim 1, wherein the generated and locally-stored pre-estimated thermodynamic properties include any combination of one or more of enthalpy of formation in liquid phase at given temperature; and liquid phase heat capacity at given temperature.

14. The method of claim 1, wherein the defined set of permissible reactions includes any combination of one or more of: saturate a benzene ring in thiophenics with 3 H2; saturate a benzene ring in pyridinics or pyrrolics with 3 H2; saturate a benzene ring in pure hydrocarbon with 3 H2; saturate an isolated thiophenic ring with 2 H2; saturate a pyridinic ring fused with a benzene ring with 2 H2, or saturate an isolated pyrrolic ring with 2 H2; saturate a benzene ring in pure hydrocarbon with 2 H2; saturate a thiophenic ring fused with a benzene ring with 1 H2; saturate a pyrrolic ring fused with a benzene ring with 1 H2; naphthenics ring opening; paraffin hydrocracking; paraffin isomerization; desulfurization of dibenzothiophene structures; desulfurization of saturated thiophenics in saturated dibenzothiophene structures; desulfurization of saturated benzothiophene, or desulfurization of saturated thiophene structures; denitrogenation of saturated nitrogen rings in saturated carbazole structures; denitrogenation of saturated nitrogen rings in saturated indoles, pyrroles, pyridine, or quinine structures; dealkylation; and inter-core linkage cracking (ILCR).

15. The method of claim 1, wherein the defined set of permissible reactions includes paraffin isomerization.

16. The method of claim 1, wherein the defined set of permissible reactions includes aromatic ring condensation.

17. The method of claim 1, wherein the defined set of permissible reactions includes any combination of one or more of: aromatic paraffin hydrogenolysis; denitrogenation of saturated nitrogen rings in saturated indoles, pyrroles, pyridine, carbazole, or quinine structures; desulfurization of sulfides; desulfurization of thiophene structures; and aromatic ring condensation.

18. The method of claim 1, wherein automatically coding a set of reaction rate equations comprises parsing a reaction into reactants, products, and stoichiometric coefficients for the reactants and products.

19. The method of claim 1, wherein automatically coding a set of reaction rate equations comprises generating one or more of a residual, a sparsity, and an analytical Jacobian.

20. The method of claim 1, wherein generating the locally stored pre-estimated thermodynamic properties is by solving one or more equations of mass balance, energy balance, momentum balance, and kinetic rate and their associated ordinary differential equations.

21. The method of claim 20, wherein solving one or more equations of mass balance, energy balance, momentum balance, and kinetic rate and their associated ordinary differential equations further comprises providing initial solutions of the one or more equations.

22. The method of claim 1, further comprising outputting a table of results of the formed model to a flowsheet simulator.

23. The method of claim 1, wherein the formed model is a full reactor model, the method further comprising:
vi) creating a list of active species of the defined set of homologous series of compounds; and
vii) storing active species of the defined set of permissible reactions in the created list of active species, thereby creating a reduced reactor model from the full reactor model.

24. A computer system for modeling chemical reactions in a chemical reactor, the computer system comprising:
one or more processors operatively coupled to associated memory, the one or more processors configured to:
i) represent reactor compounds by defining a set of homologous series of compounds in the reactor, each homologous series within the set comprising a molecular type and a carbon number, the carbon number having an upper bound and a lower bound range;
ii) define a set of permissible reactions for the defined set of homologous series of compounds;
iii) define properties of the reactor compounds;
iv) generate and locally-store pre-estimated thermodynamic properties based on the defined set of homologous series of compounds;
v) automatically code a set of reaction rate equations in equation-oriented format based on the defined set of homologous series of compounds, the defined set of permissible reactions, the defined properties of the reactor compounds, and the generated and locally-stored pre-estimated thermodynamic properties,
the automatically coded set of reaction rate equations forming a model of chemical reactions in the chemical reactor.

25-27. (canceled)

28. The computer system of claim 24, wherein the automatically coded set of reaction rate equations include Langmuir-Hinshelwood-Hougen-Watson (LHHW) rate laws.

29. The computer system of claim 24, wherein the one or more processors generate and locally store the pre-estimated thermodynamic properties by solving equations of kinetic rate including constraining kinetic rate constants by a linear free energy relationship (LFER).

30. The computer system of claim 24, wherein the automatically coded set of reaction rate equations comprises one or more of residuals, sparsity patterns, and analytical Jacobians in equation-oriented format.

31. (canceled)

32. The computer system of claim 24, wherein the defined properties of reactor compounds include any combination of one or more of: total number of carbon atoms, total number of hydrogen atoms, total number of aromatic rings, total number of naphthenic rings, total number of thiophenic rings, total number of pyrrolic rings, total number of pyridenic rings, total number of sulfur atoms, total number of nitrogen atoms, total number of oxygen atoms, standard Gibbs free energy of formation, and standard enthalpy of formation.

33. (canceled)

34. The computer system of claim 24, wherein the generated and locally-stored pre-estimated thermodynamic properties include any combination of one or more of: enthalpy of formation in gas phase at given temperature; Gibbs free energy of formation in gas phase at given temperature; gas phase heat capacity at given temperature; entropy in gas phase at given temperature; heat of vaporization; enthalpy of formation in liquid phase at given temperature; and liquid phase heat capacity at given temperature.

35-40. (canceled)

41. The computer system of claim 24, wherein automatically coding a set of reaction rate equations comprises parsing a reaction into reactants, products, and stoichiometric coefficients for the reactants and products.

42. The computer system of claim 24, wherein automatically coding a set of reaction rate equations comprises generating one or more of a residual, a sparsity, and an analytical Jacobian.

43. The computer system of claim 24, wherein the one or more processors generate the locally stored pre-estimated thermodynamic properties by solving one or more equations of mass balance, energy balance, momentum balance, and kinetic rate and their associated ordinary differential equations.

44. The computer system of claim 43, wherein solving one or more equations of mass balance, energy balance, momentum balance, and kinetic rate and their associated ordinary differential equations by the processors further comprises providing initial solutions of the one or more equations.

45. The computer system of claim 24, further comprising one of the processors outputting a table of results of the formed model to a flowsheet simulator.

46. The computer system of claim 24, wherein the formed model is a full reactor model, the one or more processors further configured to:
vi) create a list of active species of the defined set of homologous series of compounds; and
vii) store active species of the defined set of permissible reactions in the created list of active species, thereby creating a reduced reactor model from the full reactor model.

47. A computer program product comprising:
a computer readable medium carrying instructions that model chemical reactions in a chemical reactor;
the instructions include computer code which when executed by a digital processor cause a simulator of the chemical reactor to:
i) represent reactor compounds by defining a set of homologous series of compounds in the reactor, each homologous series within the set comprising a molecular type and a carbon number, the carbon number having an upper bound and a lower bound range;
ii) define a set of permissible reactions for the defined set of homologous series of compounds;
iii) define properties of the reactor compounds;
iv) generate and locally-storing pre-estimated thermodynamic properties based on the defined set of homologous series of compounds;
v) automatically code a set of reaction rate equations in equation-oriented format based on the defined set of homologous series of compounds, the defined set of permissible reactions, the defined properties of the reactor compounds, and the generated and locally-stored pre-estimated thermodynamic properties,
the automatically coded set of reaction rate equations forming a model of chemical reactions in the chemical reactor.
